(19) United States
(12) Patent Application Publication
(10) Pub. No.: US 2008/0299563 A1
(43) Pub. Date: Dec. 4, 2008

(54) HUMAN MAP2 GENE PROMOTER

(76) Inventors: Vijayasaradhi Setaluri, Madison, WI (US); Kumar Megur Ramakrishna Bhat, Manipal (IN)

Correspondence Address: QUARLES & BRADY LLP, 411 E. WISCONSIN AVENUE, SUITE 2040, MILWAUKEE, WI, 53202-4497, US

(21) Appl. No.: 12/056,556

(22) Filed: Mar. 27, 2008

Related U.S. Application Data
(60) Provisional application No. 60/908,752, filed on Mar. 29, 2007.

(51) Int. Cl. C12Q 1/68; C12N 15/11; C12N 15/00; C12P 21/04; C12N 5/06

(52) U.S. Cl. 435/6; 536/23.5; 435/320.1; 435/368; 435/69.1

(57) ABSTRACT

The human MAP2 gene promoter as well as various fragments thereof are disclosed. Nucleic acids and host cells that contain the promoter sequences are also disclosed. Further disclosed are various methods involving the use of these sequences.

### CROSS-REFERENCE TO RELATED APPLICATION

This application claims priority from U.S. provisional patent application Ser. No. 60/908,752 filed Mar. 29, 2007.

### STATEMENT REGARDING FEDERALLY SPONSORED RESEARCH OR DEVELOPMENT

This invention was made with United States government support awarded by the following agency: NIH AR048913. The United States government has certain rights in this invention.

### BACKGROUND OF THE INVENTION

Microtubule-associated protein 2 (MAP2) is a neural specific protein that stabilizes microtubules. Microtubule dynamics is regulated by a family of proteins known as MAPs. In addition to MAP2 and isoforms thereof, MAP family also includes several isoforms of MAP1 and tau, which are also expressed primarily in neurons. Among neuronal MAPs, MAP2 expression is considered a hallmark of neuronal differentiation (1,2). MAP2 is found primarily in the dendritic extensions of post-mitotic, terminally differentiated neurons. MAP2 plays a critical role in neurite outgrowth and dendrite development (3-5). Change in the expression of MAP2 is a helpful diagnostic and prognostic feature in various neurological disorders (6-8). Despite its extensive use as a marker of neuronal differentiation and its role in morphological and functional differentiation of neurons, little is known about the regulation of MAP2 gene expression.

MAP2 gene is activated in certain human non-neuronal cancers that exhibit characteristics of neural differentiation (2,9,10). Cutaneous primary melanoma, a cancer that arises from neural crest-derived melanocytes, often express abundant MAP2 protein (11). In a five-year clinical follow-up study, patients whose primary tumors express abundant MAP2 had longer disease-free survival rate than those patients with weak or no MAP2 expression (12). Moreover, reactivation of endogenous MAP2 gene transcription by treatment with pharmacological compounds or expression of exogenous MAP2 by adenovirus in metastatic melanoma cells in vitro leads to cell cycle arrest, growth inhibition and apoptosis (11,12). These observations suggested that reactivation of MAP2 gene transcription is potentially a useful strategy for the treatment of metastatic melanoma. In order to regulate the expression of MAP2, it is important to understand how the MAP2 promoter region works. However, no sequence information on MAP2 promoter is currently available.

### SUMMARY OF THE INVENTION

The present invention relates to the identification and characterization of the promoter region and various fragments thereof of the human MAP2 gene. In particular, the nucleotide sequence of the promoter region is set forth in SEQ ID NO:1 (nucleotides 1-3334), which corresponds to the −2966 to +368 fragment referred to in the Example below. In a preferred embodiment, one could remove up to 166 nucleotides from the 5′ end of the promoter and up to 358 nucleotides from the 3′ end.

In one aspect, the present invention relates to an isolated nucleic acid containing a promoter sequence selected from (a) nucleotides 1 to 3334 of SEQ ID NO:1, (b) a functional fragment of (a), (c) a nucleotide sequence that is at least 95%, 97%, 98%, or 99% identical to (a) or (b), and (d) a full length complement of (a), (b), or (c). Preferably, the nucleotide sequence of (c) retains the promoter activity of its corresponding sequence in (a) or (b). Examples of functional fragments include but are not limited to nucleotides 1852-3334 of SEQ ID NO:1 (corresponds to −1115 to +368), nucleotides 2698-3334 of SEQ ID NO:1 (corresponds to −269 to +368), and nucleotides 2698-2996 of SEQ ID NO:1 (corresponds to −269 to +30). Other examples of functional fragments include nucleotides 1-2966 of SEQ ID NO: 1 (corresponds to −2966 to −1), nucleotides 1852-2966 of SEQ ID NO:1 (corresponds to −1115 to −1), and nucleotides 2698-2966 of SEQ ID NO: 1 (corresponds to −269 to −1). Other examples of functional fragments include nucleotides 2257-3334 of SEQ ID NO:1 (corresponds to −710 to +368), nucleotides 2257-2996 of SEQ ID NO:1 (corresponds to −710 to +30), nucleotides 2257-2966 of SEQ ID NO:1 (corresponds to −710 to −1), nucleotides 2609-2996 of SEQ ID NO:1 (corresponds to −358 to +30), and nucleotides 2609-2966 of SEQ ID NO:1 (corresponds to −358 to −1). Other functional fragments can be readily determined by a skilled artisan.

In another aspect, the present invention relates to a nucleic acid that contains nucleotides 1-3334 of SEQ ID NO:1 or a functional fragment thereof as described above and a heterologous reporter gene operably linked to the sequence. The nucleic acid can be an expression vector and can be provided in a host cell.

Other aspects of the invention relate to methods of screening for agents that may alter the activity of a promoter sequence described above, methods of screening for agents that can modulate the activity of human MAP2 promoter through an N box or an E box, methods of determining whether a fragment of SEQ ID NO:1 can drive transcription under specific conditions, methods of determining which region of SEQ ID NO:1 interacts with an agent that is known to be able to alter the promoter activity of SEQ ID NO:1, and methods of using a promoter sequence described above to expressing a DNA of interest in a cell.

### BRIEF DESCRIPTION OF THE DRAWINGS

FIG. 1 (A) is a schematic diagram of the 3.3 kb human MAP2 genomic DNA fragment containing 5′ regulatory region, the first exon and partial sequence of the first intron, and different promoter constructs used in the Example below. The relative location (not drawn to scale) of putative E- and N-boxes, and the transcription start site (arrow at +1) are shown. FIG. 1 (B) shows the mouse and rat genomic sequences homologous to the 3.3 kb human MAP2 promoter region (retrieved from GenBank database by BLAST search) and conserved E- and N-boxes region (aligned using CLUSTALW sequence alignment program and shown by shading using Boxshade program). Dashes show gaps in the sequences. Conserved E boxes and N2 box sequences are highlighted.

FIG. 2 shows MAP2 promoter activity: activity of MAP2 promoter in PC12 (A), NT2/D1 (B) and melanoma cells (C). (A) Control and NGF-treated rat PC12 cells and HeLa cells were transfected in triplicates in 24-well plates with the indicated MAP2 promoter-luciferase plasmids and the Renilla luciferase plasmid. Firefly and Renilla luciferase activities were measured using Dual Luciferase Reporter Assay System (Promega). Data from a representative of at least three experiments are shown as fold induction compared to the activity of promoter without NGF. Panels on right in (A) show immunostaining of control and NGF-treated PC12 cells with anti-MAP2 mAb. (B) Human teratocarcinoma cell line NT2/D1 (grown long term in medium containing serum) and HeLa cells were transfected with the indicated MAP2 promoter constructs and luciferase activity was measured as described above. Data are shown as RLA (RLA=1). Error bars: means ±SEM. Inset in (B) shows RT-PCR analysis of MAP2 mRNA expression in HeLa and NT2/D1 cells. Total RNA (100 ng) was used to perform RT-PCR (OneStep RT-PCR kit; Qiagen) using primers that amplify a 709 bp fragment that spans human MAP2 exons 3-9. (C) shows the activity of phMAP2-2 in human primary and metastatic melanoma cell lines. Luciferase activity was measured and data (means ±SEM) are shown as RLA.

FIG. 3 shows human MAP2 promoter activity in vivo. (A) shows the whole-mount in situ staining of 11.5-day-old pHSF-6/MAP2-1/lacZ promoter transgenic embryo with X-gal for β-galactosidase gene expression. The β-galactosidase expression is seen mostly in the developing central nervous system. TV, telencephalic vesicle; VM, ventral mesencephalon; I, isthmus (midbrain-hind brain junction) and rhombic lip; and NT, neural tube. Areas of the head and the tail shown in (B) are indicated by red colored squares. (B) Paraffin sections of head region and tail regions of 11.5-day-old littermate control mouse embryos were stained with anti-MAP2ab mAb M13 (Zymed) (right panels) or control IgG (left panels) using citrate buffer antigen retrieval method followed by anti-mouse IgG-HRP and AEC detection method. Endogenous MAP2ab protein expression is seen in regions of the embryo that correspond to the region of X-gal staining. Upper panels: low magnification; lower panels: high magnification of the areas indicated by red squares in the upper panels.

FIG. 4 shows the results of the experiments for studying the role of NeuroD/BETA2 in regulation of MAP2 promoter. (A) HeLa cells and metastatic melanoma cells c22a were transfected with indicated MAP2 promoter-reporter plasmids and either 25 or 100 ng of NeuroD expression plasmid pcDNA3-NeuroD1-FLAG or 100 ng of pcDNA3. Equal amount of total DNA was used in all transfection. Luciferase activities were measured and RLA was calculated as described in above in connection with FIG. 1. Data shown are means ±SEM. (B) show electrophoretic mobility shift analysis of E-box sequence. ³²P-labeled double-stranded 22mer oligonucleotides with core sequence of E9 box CAGATG (see Materials and Methods) were incubated with nuclear extract (5-10 μg protein) of c22a metastatic melanoma cells transfected with pNeuroD either alone or in the presence of 5-50 M excess cold E9, E10 or mutant E9 oligonucleotides. Lanes 8 and 9 show mobility shift pattern after the addition of control rabbit IgG (lane 8) or rabbit anti-NeuroD antibody (lane 9) to the E8 oligonucleotide-protein complexes.

FIG. 5 shows the indirect activation MAP2 promoter by NeuroD. (A) HeLa cells and metastatic melanoma cells c22a were co-transfected with phMAP2-3 promoter-reporter plasmid lacking the E9 box and NeuroD expression plasmid pcDNA3-NeuroD-FLAG (pNeuroD) (25 and 100 ng) or 100 ng of control pcDNA3 vector. (B) shows the effect of mutation of the core E9 box sequence (CAGATG→ACGAGT) on MAP2 promoter activity. Asterisk indicates P-value (Student's t-test, P<0.001). (C) shows the dose-dependent activation of mutE9-phMAP2-2 promoter by NeuroD. c22a melanoma cells were co-transfected with mutE9 phMAP2-3 and increasing amount of pNeuroD plasmid. Equal amounts of total DNA were used in all transfections. Luciferase activities were measured and RLA (means ±SEM) was calculated as described above in connection with FIG. 1.

FIG. 6 shows that HES1 (hairy and enhancer of split) represses MAP2 promoter activity. (A) Human metastatic melanoma cells were transfected with 1.4 kb phMAP2-2 promoter and increasing amounts (0.1, 0.3 and 0.6 μg) of mouse Hes1 expression plasmid pCI-Hes1 or 0.6 μg the empty vector pcDNA3. Forty-eight hours after transfection luciferase activities were measured. RLA were calculated and MAP2 promoter activity in Hes1 expressing cells is shown as percent (means ±SEM) of pcDNA3 co-transfected cells. (B) shows mutational analysis of the role of N-boxes in repression of MAP2 promoter by Hes1. Metastatic melanoma cells were transfected with wild-type (wt) and N-box mutated (mutated nucleotides shown underlined) phMAP2-2 promoter plasmids mutN1 (ACCAGA), mutN2 (GCGCC) and mutN1N2 and luciferase activity in cells transfected with mutant plasmids is shown as percent of wild-type transfected cells (*P<0.05 compared with wt; one-way ANOVA). (C) Metastatic melanoma cells were transfected with wt and N-box mutant plasmids together with 1.5 μg empty vector pcDNA or pCI-Hes1 plasmid and luciferase activities in cell lysates was measured 48 h after the transfection. Repression of the wild-type and N-box mutant promoter activity by Hes1 is shown as percent activity of wild-type and the mutant plasmids in cells transfected with the pcDNA empty vector (*P<0.05; **P<0.01 compared with wt; one-way ANOVA). (D) shows repression of the phMAP2-4 (−269/+30) promoter by Hes1. Melanoma cells in 24-well plate were transfected, in triplicates, with 0.6 μg phMAP2-4 plasmid and 0.6 μg of empty vector pcDNA or indicated amounts of pCI-Hes1 plasmid. After 48 h transfection, cells were lysed and RLA was determined. MAP2 promoter activity in the presence of Hes1 is shown as percent of empty vector transfected cells. Equal amount of total DNA was used in all transfection. Data (mean ±SEM) shown are representative of least three experiments. (E) shows electrophoretic mobility shift analysis of N-box. ³²P-labeled N2 box double-stranded oligonucleotides were incubated with nuclear extract (5-10 μg protein) of pCI-Hes1 transfected c22a metastatic melanoma cell either alone or in the presence of 5-50 M excess cold N1, N2 or mutant N2 box oligonucleotides or a polyclonal Hes1 antibody or a control rabbit IgG. Left arrow indicates the specific N2 box DNA-protein complex and the right arrow indicates the Hes1 supershifted complexes. Asterisks indicate non-specific complexes.

FIG. 7 shows that HES1 is a dominant repressor of MAP2 promoter activity. (A) Melanoma cells were transfected with phMAP2-2 promoter-luciferase plasmid alone, or with Hes1 expression plasmid pCI-Hes1 or NeuroD expression plasmid pcDNA3-NeuroD-FLAG. In addition, 0.1 and 0.3 μg of Hes1 expression plasmid pCI-Hes1 was included in cells co-transfected with NeuroD. (B) c22a melanoma cells were transfected with phMAP2-2 promoter-luciferase plasmid alone, or with Hes1 expression plasmid pCI-Hes1 or NeuroD expression plasmid pcDNA3-NeuroD-FLAG. Cells in additional wells transfected with pCI-Hes1 also received 0.1 or 0.3 μg of pNeuroD plasmid. Equal amount of total DNA was used in all transfection. Luciferase activities were measured and the promoter activity in cells co-transfected with either pcDNA3-NeuroD or pCI-Hes1 alone or with increasing amounts of pCI-Hes1 or pcDNA3-NeuroD1, respectively, is shown as percentage (means ±SEM) of activity in cells transfected with phMAP2-2 and pcDNA. (C) shows the expression of NeuroD and HES1 in melanocytes and melanoma cells. Western blot analysis of NeuroD expression in control and pcDNA3-Neuro-FLAG plasmid transfected COS cells, neonatal foreskin melanocytes (NMC), primary melanoma cell line WM75 and various metastatic melanoma cell lines. Seventy-five micrograms of detergent soluble total cellular proteins were separated by SDS-PAGE and immunoblotted with polyclonal goat anti-mouse NeuroD (upper panel) and anti-mouse Hes1 antibody (lower panel), followed by HRP-conjugated secondary antibody. Protein bands were detected by chemiluminescence. Western blotting for α-tubulin is shown as a control for protein loading. (D) shows the results of chromatin immunoprecipitation assay. Sheared chromatin from melanoma cells was immunoprecipitated (IP) with appropriate antibody (anti-HES1, anti-NeuroD or control IgG) and antibody bound DNA was isolated according to the manufacturer's protocol (Active Motif, Carlsbad, Calif.). Immunoprecipitated DNA was used as template in PCR using N1 and N2 primer (for HES1), E9-E11 primer (for NeuroD). GAPDH primers were used as control.

FIG. 8 shows that Notch1 signaling is involved in MAP2 promoter regulation in melanoma cells. Primary melanoma cells, WM35 (A) and metastatic melanoma cells, 451 Lu (B) were either pre-treated with 1 or 2 μM γ-secretase inhibitor (DAPT) or transfected with 0.6 μg of HES1shRNA and then treated with 2 μM DAPT. After 24 h, the cells were transfected with phMAP2-2 luciferase-promoter construct. Cells were lysed and luciferase activity was measured 48 h after promoter transfection. RLA was calculated as described above in connection with FIG. 2. Data are shown as percent activity of control DMSO-treated cells or control shRNA transfected cells, and are from a representative of two individual experiments with triplicates. (C) shows the Western blotting analysis of primary (WM35) and metastatic (451Lu) melanoma cells transfected with HES1shRNA alone or in combination with the treatment of DAPT. A decreased level of endogenous HES1 protein was observed. Western blotting of β-actin is shown as loading control.

### DETAILED DESCRIPTION OF THE INVENTION

The term “isolated nucleic acid” used herein means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than two separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of clones, e.g., as these occur in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine.

As used in this application, “percent identity” between nucleotide sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993), or other methods. The noted algorithm is incorporated into the NBLAST program of Altschul et al. (J. Mol. Biol. 215, 403-410, 1990). BLAST nucleotide searches are performed with the NBLAST program, score=100, wordlength=12, to obtain nucleotide sequences homologous to a polynucleotide of the invention. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., NBLAST) are used.

We have cloned the promoter region of human MAP2 gene and found that the promoter region as well as various fragments thereof were sufficient for neuronal-specific expression both in vitro and in vivo. The cloned promoter region contains several E- and N-boxes, which are binding sites for basic helix-loop-helix (bHLH) transcription factors. We found that MAP2 promoter is activated by neurogenic bHLH factor NeuroD/BETA2 and repressed by the bHLH repressor Hairy and Enhancer of Split 1 (HES1), a regulator of neuronal differentiation. Our study suggests that MAP2 gene is regulated by the relative levels of NeuroD and HES1, and predominantly by the level of HES1 protein. Human MAP2 promoter sequences and associated promoter activities are set forth in SEQ ID NO:1 and the Example below. The disclosure here enables new tools to study MAP2 expression and to screen for agents that may modulate the expression of MAP2. Such agents may be useful for inducing neuronal differentiation and for treating melanoma.

Cells that support the activity of human MAP2 promoter include but are not limited to (1) differentiated neural cells of either the central or peripheral nervous system such as neurons (e.g., retinal ganglion cells), astrocytes, and oligodendrocytes, (2) neural stem cells and neural progenitor cells that are derived from embryonic or adult stem cells and are at various stages of neural differentiation, (3) cells from benign and malignant tumors of the central or peripheral nervous system such as glioblastoma cells, neuroblastoma cells, glioma cells, and oligodendroma cells, (4) germ cell tumor cells such as embryonal carcinoma or teratocarcinoma cells, (5) endocrine and neuroendocrine cells such as pancreatic cells, adrenal medullary cells, and neuroendocrine cells of respiratory and gastrointestinal systems, (6) cells of neuroendocrine tumors (e.g., pheochromocytoma) and other tumors that show neuronal or neuroendocrine differentiation (e.g., melanoma, pancreatic cancer, non-small cell lung cancer, breast cancer, and prostate cancer cells). It is noted that when neural stem/progenitor cells or cells of various types of tumors described above are used, they may need to be maintained under conditions suitable for inducing them to differentiate into neuron-like cells in order to support the promoter activity of human MAP2 promoter. For example, as shown in the Example below, rat pheochromocytoma PC12 cells need to be treated with NGF and human teratocarcinoma NT2/D1 cells need to be treated with retinoids or retinoic acid.

In one aspect, the present invention relates to an isolated nucleic acid containing a promoter sequence selected from (a) nucleotides 1 to 3334 of SEQ ID NO:1, (b) a functional fragment of (a), (c) a nucleotide sequence that is at least 95%, 97%, or 99% identical to (a) or (b), and (d) a full length complement of (a), (b), or (c). Preferably, the nucleotide sequence of (c) retains the promoter activity of its corresponding sequence in (a) or (b). In a preferred embodiment, one could remove up to 166 nucleotides from the 5′ end of the promoter and up to 358 nucleotides from the 3′ end. By “a functional fragment,” we mean that the fragment contains at least one activator or repressor for gene expression under specific conditions. An activator functions to drive gene expression to above a background level and a repressor functions to bring the expression back toward the background level. If both activators and repressors are present, the actual expression level will be determined by the combined effects of the activators and repressors. The background level is defined as the expression level in the absence of any promoter sequence or in the presence of an unrelated nucleotide sequence with no promoter activity. For the purpose of the present invention, a functional fragment is at least 50 nucleotides long and preferably at least 100, 200, 500, or 1,000 nucleotides long. It should be noted that all functional fragments described above are useful in the screening method and other methods provided below. It is not required that the functional fragment drives the expression to above a background level. It is only necessary that perturbation of the fragment's function can be measured. Examples of functional fragments include but are not limited to nucleotides 1852-3334 of SEQ ID NO:1 (corresponds to −1115 to +368), nucleotides 2698-3334 of SEQ ID NO:1 (corresponds to −269 to +368), and nucleotides 2698-2996 of SEQ ID NO:1 (corresponds to −269 to +30). Other examples of functional fragments include nucleotides 1-2966 of SEQ ID NO:1 (corresponds to −2966 to −1), nucleotides 1852-2966 of SEQ ID NO:1 (corresponds to −1115 to −1), and nucleotides 2698-2966 of SEQ ID NO:1 (corresponds to −269 to −1). Other examples of functional fragments include nucleotides 2257-3334 of SEQ ID NO:1 (corresponds to −710 to +368), nucleotides 2257-2996 of SEQ ID NO:1 (corresponds to −710 to +30), nucleotides 2257-2966 of SEQ ID NO:1 (corresponds to −710 to −1), nucleotides 2609-2996 of SEQ ID NO:1 (corresponds to −358 to +30), and nucleotides 2609-2966 of SEQ ID NO:1 (corresponds to −358 to −1). Other functional fragments can be readily determined by a skilled artisan using well known techniques such as those described below and in the Example.

In another aspect, the present invention relates to a nucleic acid (which can but does not have to be an expression vector) that contains a promoter sequence disclosed herein (which can be a functional fragment described above) and a heterologous reporter gene operably linked to the promoter sequence. Such a nucleic acid is useful in many of the methods described below. The term “reporter gene” is defined here to encompass any polynucleotide the transcription of which under the control of a promoter sequence, the subsequent translation thereof, or both can be readily detected by a skilled artisan. Thus, the reporter gene does not have to encode a full-length protein. In some instances, the reporter gene can even be an oligonucleotide. In one embodiment, the reporter gene is a polynucleotide that encodes a protein with a detectable activity.

In another aspect, the present invention relates to a cultured cell that contains a nucleic acid described above. In one embodiment, the cell is selected from differentiated neural cells, neural stem cells and neural progenitor cells, cells from nervous system tumors, and germ cell tumor cells.

In another aspect, the present invention relates to a method for screening for an agent that can alter the promoter activity of a promoter sequence disclosed herein and such agents are useful for modulating MAP2 expression. The method involves providing a nucleic acid that contains a promoter sequence disclosed herein operably linked to a reporter gene. The nucleic acid is next exposed to conditions suitable for the promoter sequence to drive the expression of the reporter gene. Two groups of nucleic acids can be set up here. In one group, the expression of the reporter gene is measured in the presence of a test agent. In the other group (control group), the expression is measured in the absence of the test agent. The expression of the reporter gene in both groups can then be compared. A higher or lower expression in the test agent group than in the control group indicates that the agent may alter the activity of the promoter sequence. In one embodiment, the method is used to screen for an agent that can increase the activity of a human MAP2 promoter sequence disclosed herein.

A skilled artisan is familiar with the assay systems that can be used for measuring the expression of a reporter under the control of a promoter sequence and the present invention is not limited to any particular assay system. For example, an expression vector containing a promoter sequence and a luciferase reporter gene can be introduced into suitable host cells and the expression of the reporter gene under suitable conditions can be measured by the luciferase activity. It is understood that other reporter genes can also be used. Furthermore, the expression of the reporter gene can also be measured at the mRNA level or at the protein level with a method other than assaying the enzyme activity. For instance, the amount of a reporter gene product can be measured by the use of an antibody specific for the product using an ELISA assay. Examples of suitable host cells have been described above.

In another aspect, the present invention relates to a method for screening for an agent that can alter the activity of human MAP2 promoter through an N box or an E box, or a method for determining whether an agent that is known to be able to alter the human MAP2 promoter activity alters the activity through an N box or an E box. The method involves providing a first human MAP2 promoter sequence (preferably at least 50 nucleotides in length) that contains at least one N box or E box, providing a second human MAP2 promoter sequence (preferably at least 50 nucleotides in length) that is the same as the first sequence except that the N Box or E box is mutated, and determining and comparing the effect of the agent on the promoter activity of the first and second sequences wherein the presence of an effect on the first but not the second sequence indicates that the agent can alter the human MAP2 promoter activity through an N box or an E box. The effect of the agent on the promoter activity of the first and second sequences can be determined as described above by employing nucleic acids that contain the first or the second sequence and a reporter gene. Although substitution mutations of N and E boxes were employed in the Example below, other mutations such as insertion and deletion mutations can also be used in the method.

In another aspect, the present invention relates to a method for determining whether a fragment of SEQ ID NO:1 (nucleotides 1-3334), preferably at least 50 nucleotides in length, is functional. The method includes the steps of providing a nucleic acid that contains the fragment and a heterologous reporter gene operably linked to the fragment, measuring the expression level of the reporter gene under suitable conditions, and comparing the expression level to a control under the same conditions. The control can be a negative control, by which we mean the background expression level as defined above. The control can also be a positive control, by which we mean the expression level of a reporter gene driven by nucleotides 1-3334 of SEQ ID NO:1 or a known functional fragment thereof. Preferably, the method is used to identify a fragment of SEQ ID NO:1 that can drive the expression above said background level. In one embodiment, the expression of the reporter gene is assayed in a cell as described above. An isolated nucleic acid containing a functional fragment identified by the method, a nucleic acid containing the functional fragment operably linked to a heterologous reporter gene, and a host cell containing such a nucleic acid are also within the scope of the present invention. Also within the scope of the present invention is a method of using the functional fragment identified to screen for agents that may alter the activity of the fragment as described above.

In another aspect, the present invention relates to a method of determining which region of SEQ ID NO:1 (nucleotides 1-3334) interacts with an agent known to be able to alter the promoter activity of SEQ ID NO:1. The method involves providing multiple groups of nucleic acids in which a reporter gene is operably linked to a fragment of SEQ ID NO:1 (preferably at least 50 nucleotides in length) and wherein the nucleic acid of the same group contain the same fragment and the nucleic acids in different groups contain different fragments. The nucleic acids are next subjected to conditions suitable for the fragments to drive the expression of the reporter gene. The expression of the reporter gene in the absence and presence of the agent is then measured and compared, and the effects of the agent on the promoter activity of different fragments are determined. Finally, the effects of the agent on the promoter activity of different fragments are compared and the region of SEQ ID NO:1 that interacts with the agent can be identified. In one embodiment, the expression of the reporter gene is assayed in a cell as described above.

In another aspect, the present invention relates to a method for expressing a DNA sequence by providing in a cell a DNA construct that contains a promoter sequence of the present invention operably linked to a heterologous DNA sequence of interest and subjecting the cell to conditions that allow the expression of the heterologous DNA. For example, a DNA sequence of interest can be provided in an expression vector wherein the DNA sequence is operably linked to a promoter sequence of the present invention. The vector can then be introduced into a suitable host cell by transfection and the host cell can be maintained in culture to allow the expression of the DNA sequence, which can be at the mRNA level or polypeptide level. Examples of suitable host cells have been described above.

The invention will be more fully understood upon consideration of the following example, which is not intended to limit the scope of the invention.

### EXAMPLE

### Materials and Methods

Cell culture: Mouse neuroblastoma cell line, Neuro2a, and human embryonal carcinoma cell line NT2-D1 were purchased from American Type Culture Collection (Manassas, Va.). Neuro2a cells were grown in minimal essential medium (MEM). The human glioblastoma cell lines U251, U138 and rat PC12 cells were cultured in Rosewell Park Memorial Institute (RPMI) medium and DMEM, respectively. The human breast cancer cell line MCF-7 was cultured in MEM supplemented with bovine insulin. Human fibroblast cell line NIH3T3 was cultured in DMEM with high glucose, and human embryonic teratocarcinoma cell line NT2-D1 was cultured in DMEM with high glucose and supplemented with NaHCO₃ (1.5 g/l). Neonatal foreskin melanocytes, primary melanoma cell lines WM35 and WM75, and metastatic melanoma cell lines SK-MEL-19, 451Lu and SK-MEL-23 c1.22a were cultured as described in (16), which is herein incorporated by reference in its entirety. All culture media were supplemented with 10% fetal bovine serum (FBS). Medium for PC12 cells were supplemented with 5% FBS and 10% horse serum, 1% L-glutamine, and 1% penicillin and streptomycin. All cell culture media, L-glutamine, penicillin and streptomycin, non-essential amino acids were from GIBCO (Grand Island, N.Y.), and FBS was from Sigma (St Louis, Mo.) or Atlanta Biologicals (Lawrenceville, Ga.).

Differentiation of PC12 cells: Before differentiation, cells were grown in 1% horse serum containing media on collagen-coated tissue culture dishes. After the cells got attached, they were treated with 100 ng/ml nerve growth factor (NGF 2.5S; Promega, Madison, Wis.). Twenty-four hours after NGF treatment, cells were transfected with promoter constructs and cultured for additional 48 h in medium containing NGF. For MAP2 immunostaining, PC12 cells were treated with 100 ng/ml NGF for 6 days (17,18).

Plasmids and antibodies: pCI-Hes1, a mouse Hes1 expression plasmid and Hes1 antibody (19) were gifts from Prof. R. Kageyama (Institute for Virus research, Kyoto University, Kyoto, Japan). Rabbit polyclonal antibody directed against the C-terminal region of rat Hes1 for supershift experiments was kindly provided by Dr Y. N. Jan (University of California, San Francisco, Calif.). NeuroD expression plasmid pCDNA3-NeuroD1-FLAG (pNeuroD) (20) was provided by Dr Haeyoung Suh-Kim (Ajou University School of Medicine, Suwon, Korea). Goat polyclonal anti-NeuroD antibody for western blot analysis (N-19) and for supershift experiments and chromatin immunoprecipitation assay (N-19X) and anti-HES1 antibody for ChIP (N-17X) were purchased from Santa Cruz Biotech (Santa Cruz, Calif.). Affinity purified anti-MAP2ab was obtained from Zymed Laboratories (San Francisco, Calif.), anti-a-tubulin antibody was obtained from Sigma (St Louis, Mo.). Horseradish peroxidase (HRP) conjugated goat anti-mouse IgG was from Jackson Laboratories (West Grove, Pa.). Anti-mouse IgG-FITC was purchased from Dako Corp. (Carpentaria, Calif.).

Construction of human MAP2 promoter-luciferase plasmids: Human MAP2 genomic sequence (GenBank accession no. NT_{—}005403) was identified using BLAST search with MAP2 cDNA sequence, and a 1.4 kb region corresponding −1115 to +368 was amplified by PCR using forward primer 5′-CTGGCCTTTTTGGTTCTCAT (SEQ ID NO:2) and reverse primer 5′-TAGTCTAAGCTTAGC TGAGAATCTACCGA (SEQ ID NO:3) containing HindIII sites and subcloned into the HindIII site of pGL3 basic vector. This plasmid is designated as phMAP2-2. Since attempts to amplify the longer genomic DNA fragment containing region upstream of −1115 were unsuccessful, we first PCR amplified a region corresponding to −2966 to −106 using a forward primer 5′-GACGACAAGCTTCAGAAGAAGGGTAAGGCAAGCATCA (SEQ ID NO:4) and a reverse primer 5′-GACGACAAGCTTCGCAGTGGCGAACAGGAAAA (SEQ ID NO:5), and constructed the longer phMAP2-1 plasmid by joining the 5′ −2966/−269 fragment with −269/+368 fragment at the internal KpnI site. Deletion constructs phMAP2-3 (−269/+368) were made by splicing KpnI/KpnI (−1115/−268) fragment out from phMAP2-2 and re-ligating the plasmid. phMAP2-4 (−269/+30) was made by digestion of phMAP2-3 with PstI (+31) and HindIII (+368), followed by fill-in reaction with DNA polymerase (Klenow fragment) and blunt end ligation.

E- and N-box mutant MAP2 promoters: mutE1 (−2888/−2883), mutE2 (−2799/−2794), mutE3 (−2304/−2299), mutE4 (−1593/−1588) individual mutations and various combination of E-box mutations (E1,2,3,4; E1,2,3; E2,3) were generated in phMAP2-1, mutN1 (−961/−956), mutN2 (−63/−58), mutE9 (−358/−353), mutE11 (−279/−274), mutE12 (+67/+72) and mutE13 (+267/+272) and combinations of these mutations (E12,13; E12,13,N1) were generated in phMAP2-2 using the QuikChange site-directed mutagenesis kit (Stratagene, La Jolla, Calif.) according to the manufacturer's protocol. The following primers were used for mutagenesis (only sense sequence is given and mutated nucleotides within the consensus sequence are underlined): mutE1: 5′-CACAGAAGTATCTACATGTTCGGTTGTCTTG (SEQ ID NO:6); mutE2: 5′-CTCCATGTTGGAAAGCATGCGGAAAAAAAGAAAAG (SEQ ID NO:7); mutE3: 5′-GATGATGGTGGCAGGTACACTCTTTTAGACATC (SEQ ID NO:8); mutE4: 5′-GGGAAAGAAAGTGTCTACGTGAGAGAAGAAAGG (SEQ ID NO:9); mutE9: 5′-CACAAAGGAGACGCGAGGATGACATTCATAG (SEQ ID NO: 10); mutE11: 5′-GCCTGTCAGGGCTTCCGTACCAGAAG (SEQ ID NO: 11); mutE12: 5′-GTGGTATTGCTGAATATTCGCTGGTAATGG (SEQ ID NO:12); mutE13: 5′-GAGTATAAAGAATGGGCTTGCCTTACTGGTGAG (SEQ ID NO:13); mutN1: 5′-CACTTAACCTCCCTAACCAGAGGCACTGTAAAATAG (SEQ ID NO: 14); mutN2: 5′-GGCACACACAGAGCCGCCCTGGTGGCTTGCAG (SEQ ID NO: 15).

Transient transfection and luciferase assays: Transient transfections with various promoter constructs were performed using Lipofectamine (Invitrogen, Carlsbad, Calif.) according to the manufacturer's instructions. Cells in 24- or 6-well tissue culture dishes, in triplicates, were transfected with 0.6 or 3 μg, respectively, of empty vector pGL3 or promoter-reporter constructs (phMAP2-1, phMAP2-2, phMAP2-3 and phMAP2-4) and 0.07 or 0.5 μg Renilla luciferase plasmid pRL. In co-transfection experiments, different amounts of NeuroD and Hes1 expression plasmids were added. For Notch1 inhibition and HES1 knockdown experiments, cells were either pre-treated with indicated amount of γ-secretase inhibitor-DAPT (Calbiochem, La Jolla, Calif.) and/or transfected with indicated amount of lentiviral HES1shRNA plasmid (The RNAi Consortium collection, a library of short hairpin DNAs cloned in pLKO.1 lentiviral vector; Open biosystem, Huntsville, Ala.) 1 day before transfection with MAP2 promoter constructs. After 48 h of transfection, cells were washed with pre-chilled PBS and lysed in passive lysis buffer (Dual Luciferase kit, Promega). Firefly luciferase and Renilla luciferase activities in the cell lysates were measured according to the manufacturer's instructions using TD 20/20-luminometer (Turner Biosystems, Sunnyvale, Calif.). Firefly luciferase activity was normalized to the Renilla luciferase activity and reported as relative luciferase activity (RLA).

Transient transgenic expression: The 3.3 kb MAP2 genomic fragment from phMAP2-1 and 1.4 kb fragment from phMAP2-2 was isolated by digestion with HindIII and subcloned into the HindIII site upstream of Hsp68-lacZ gene in pHSF5 vector (21). The orientation and sequences were confirmed by restriction digestion and sequencing. Transgenic embryos were generated by Biotechnology Center (University of Wisconsin, Madison, Wis.). Embryos harvested on day 11.5 were stained for β-galactosidase activity essentially as described by Shashikant et al. (21).

Immunostaining: Control embryos (11.5 days old) were fixed in 4% formaldehyde overnight at 4° C. and dehydrated using a graded series of methanol, and embedded in paraffin. Sections (9 μm thick) were cut and stained for MAP2 with anti-MAP2ab (Sigma) using citrate buffer antigen retrieval method (22) followed by anti-mouse IgG-HRP and the antigen was detected with HRP substrate diaminobenzidene.

Electrophoresis mobility shift assay (EMSA): Wild type E- and N-boxes containing oligonucleotides were labeled with [γ-32P]ATP (3000 Ci/mMol; ICN Biomedicals, Inc., Irvine, Calif.) with T4-polynucleotide kinase (Promega Corp., Madison, Wis.). Unincorporated label was removed using NAP5 columns (Amersham Pharmacia Biotech, Piscataway, N.J.). Radioactivity in the eluted fractions was measured and ˜20,000 c.p.m were used for each binding reaction. The following reverse phase purified oligonucleotides were annealed and used as probes and competitors. Core sequences of E- and N-box are underlined: WtN1, 5′-CCTCCCTACACAAGGGCACTGT (SEQ ID NO:16); mutN1, 5′-CCTCCCTAACCAGAGGCACTGT (SEQ ID NO:17); WtN2, 5′-CACACACAGACACGAGCTGGTGGCTT (SEQ ID NO:18); mutN2, 5′-CACACACAGAACCGGACTGGTGGCTT (SEQ ID NO: 19); mutN2-scrambled, 5′-ACCAACCAGAACCGGACTGGTGGCTT (SEQ ID NO:20); WtE6E7, 5′-GGGATATTCAACTGCTAATTTCAGTTGCTAACATG (SEQ ID NO:21); mutE6E7, 5′-GGGATATTACACGTCTAATTTACGTGTCTAACATG (SEQ ID NO:22); WtE8, 5′-AACATGGCCAAGTGCATAATTA (SEQ ID NO:23); mutE8, 5′-AACATGGCACAGGTCATAATTA (SEQ ID NO:24); wtE9, 5′-AAGGAGACCAGATGATGACATT (SEQ ID NO:25); mutE9, 5′-AAGGAGACACGAGTATGACATT (SEQ ID NO:26); wtE10, 5′-TGGGGACACACTTGGATG GAGG (SEQ ID NO:27); mutE10, 5′-TGGGGACAACCTGTGATGGAGG (SEQ ID NO:28); wtE11, 5′-GCCTGTCAGGCATTTGGTACCAGAAG (SEQ ID NO:29); and mutE11, 5′-GCCTGTCAGGGCTTCCGTACCAGAAG (SEQ ID NO:30). Nuclear extracts of COS-7 or c1.22a melanoma cells transfected with Hes1 expression plasmid or with NeuroD expression plasmid were prepared essentially as described previously (16). Labeled probes were incubated with 5-10 μg of nuclear extract at room temperature for 30 min. For supershift experiments, anti-Hes1 or anti-NeuroD antibody or unrelated polyclonal antibody were added to the reaction mixture and incubated at room temperature for additional 30 min. DNA-protein complexes were analyzed by electrophoresis on non-denaturing 5% polyacrylamide gels run at constant 200 V for 2 h in 0.5×TBE. The gels were dried and exposed to X-ray film (Eastman Kodak Company, Rochester, N.Y.).

Chromatin immunoprecipitation assay (ChIP assay): Assay was performed using ChIP-IT kit (Active Motif, Carlsbad, Calif.) following the manufacturer's protocol. Briefly, SK-MEL-23 cells were cross linked, chromatin was prepared and immunoprecipitated with anti-HES1 antibody (Santa Cruz) or with control IgG. Then, immunoprecipitated DNA was eluted and PCR amplified using appropriate primers. For PCR amplification of N1 (36 cycles) and N2 (29 cycles) box region in MAP2 promoter region, the following primers were used: N1-forward, 5′-CCCAGGAAATAAATGCAGGA (SEQ ID NO:31); N1-reverse, 5′-GGGCCGATATGTGATTTCTG (SEQ ID NO:32); N2-forward, 5′-CCACTCGCCTTATTTTCCTG (SEQ ID NO:33); and N2-reverse, 5′-CGCATATGCAGCAAACAC (SEQ ID NO:34). For PCR amplification of E9-E11 boxes (36 cycles) in promoter region the following primers were used: forward, 5′-TAAGCGGTGTGTGTGTGTGC (SEQ ID NO:35); and reverse, 5′-ATGATGACAAGCCACTCAGC (SEQ ID NO:36).

RT-PCR: Total RNA was isolated from different melanoma and non-melanoma cells using RNeasy Mini Kit (Qiagen, Valencia, Calif.) and amplified by using one-step RT-PCR Kit (Qiagen) as per manufacturer's instructions using 100 ng of total RNA and MAP2 forward primer 5′-GCAGTTCTCAAAGGCTAGAC (SEQ ID NO:37) (nt 31-50 in MAP2ab CDNA in exon 3) and reverse primer 5′-TTGATCGTGGAACTCCATCT (SEQ ID NO:38) (nt 721-740 in MAP2ab cDNA in exon 9).

SDS-PAGE and western blotting: Total cell lysates of normal melanocytes, different melanoma and non-melanoma cell lines were prepared using lysis buffer [1% Triton X-100 in PBS containing cocktail of protease inhibitors (Roche Diagnostics Corp., Indianapolis, Ind.)]. Protein was estimated using BCA protein assay kit (Pierce Biotechnology Inc., Rockford, Ill.) and 50-100 μg protein was separated on 9% SDS-PAGE, transferred on to Polyscreen membrane (NEN Life Sciences, Boston, Mass.) and the membranes were blocked and then incubated overnight at 4° C. with the primary antibodies diluted (anti-Hes1 at 0.6 μg/ml; anti-NeuroD at 1:250; anti-α-tubulin at 1:2000 and anti-β-actin at 1:2000) in 5% non-fat dry milk in TBST, followed by incubation with the appropriate HRP-conjugated secondary antibodies (at 1:5000 to 1: 10,000 dilution) for 1 h at room temperature. Protein bands were detected by chemiluminescence using ECL kit (Amersham Biosciences, Piscataway, N.J.).

Results

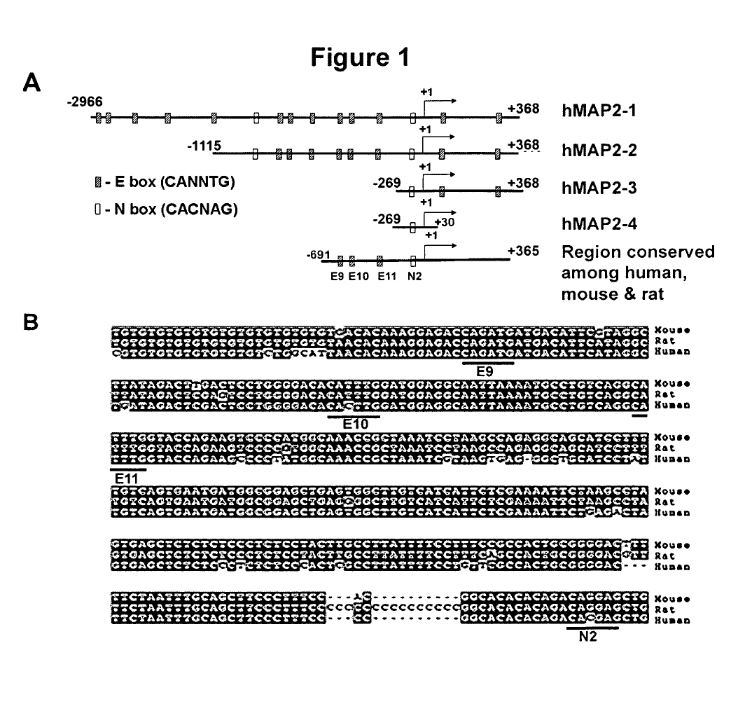

Nucleotide sequences upstream of MAP2 gene are conserved in mouse, rat and human: Using BLAST search, we identified the human genomic sequences upstream of MAP2 cDNA in a chromosome 2 contig (GenBank accession no. NT_{—}005403) and amplified, by PCR, a 3334 bp genomic DNA fragment consisting 2966 nt upstream of MAP2 transcription start site (+1), exon 1 (78 bp) and a part of intron 1 (291 bp). We sequenced this fragment (GenBank accession no. DQ 386449), and cloned it into pGL3 luciferase reporter vector (phMAP2-1; −2966/+368). First, we analyzed this MAP2 genomic sequence for transcription factor binding sites and compared the human sequence with mouse and rat MAP2 genomic sequences using CLUSTALW program (FIGS. 1A and B). This analysis showed the following:

(i) Mouse, rat (GenBank accession nos: mouse, NT_{—}039170; and rat, NW_{—}047816) and human MAP2 genomic sequences show 80% sequence identity in 1 kb region corresponding to −691/+365 of human MAP2 genomic sequence. In addition, an 87 bp rat genomic sequence showed 85% identity to human sequences between −1388/−1302. There were no homologous sequences to this 87 bp in the mouse.

(ii) Inspection of the 3.3 kb human MAP2 5′ genomic sequence showed the presence of 13 (E1-E13) putative E boxes (CANNTG) and two (N1 and N2) putative N-boxes (CACNAG), which are potential sites for binding bHLH proteins including neuronal transcriptional activators and repressors (23,24).

(iii) The proximal cluster of three E boxes (E9-E11) at −358/−353, −313/−308 and −279/−274, and a N2 box −63/−58 found in the human MAP2 sequence are conserved among mouse, rat and human sequences; additional upstream (E1-E8) and downstream (E12 and E13 within exon 1 and at +267/+272 within intron 1) E boxes and N1 box at −961/−956 within the human MAP2 promoter sequence are not present in the mouse and the rat MAP2 genomic sequences.

(iv) No canonical TATA box could be identified in the MAP2 proximal 5′ genomic sequences, suggesting that MAP2 promoter is a TATA-less promoter (20,25,26).

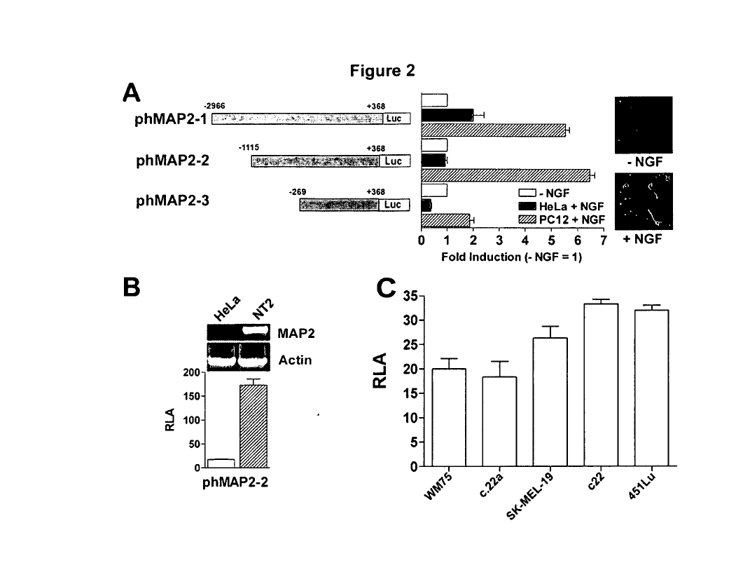

MAP2 promoter activity: To investigate the promoter activity of MAP2 5′ genomic sequence, we generated, in addition to the 3.3 kb luciferase reporter plasmid (phMAP2-1), 5′ deletion constructs phMAP2-2 (−1115/+368), phMAP2-3 (−269/+368) and a 3′ deletion construct phMAP2-4 (−269/+30) that lacks intron 1 sequence and part of exon 1. We tested the activity of these MAP2 promoter plasmids in rat PC12 cells and human teratocarcinoma cell line NT2/D1 by transient transfection followed by luciferase assays. PC12 cells treated with NGF are known to differentiate into neuron-like cells with neurite outgrowths and express MAP2 (FIG. 2A, inset) (17,18). NT2/D1 is an inducible model system for neuronal differentiation. Continuous culture of NT2/D1 cells in complete serum containing retinoids or the addition of retinoic acid to the steroid free culture medium is known to induce neuronal differentiation and MAP2 expression (as shown by RT-PCR, inset in FIG. 2B) in these cells (27).

In NGF-treated PC12 cells, phMAP2-1 and phMAP2-2 promoter constructs showed 6- to 7-fold higher luciferase activity compared with untreated cells. Although promoter construct lacking sequences between −1115/−269 (phMAP2-3) showed ˜2-fold higher basal activity compared with phMAP2-1 and phMAP2-2 in untreated PC12 cells, NGF treatment did not produce a significant increase in the luciferase activity. In non-neuronal HeLa cells, all three promoter constructs produced only weak luciferase activity and treatment with NGF produced a 2-fold increase only in cells transfected with the 3.3 kb phMAP2-1 promoter construct (FIG. 2A). We found that luciferase activity driven by the 3.3 kb MAP2-1 promoter was always lower than that of the shorter 1.4 kb MAP2-2 promoter fragment in every cell line tested, indicating the presence of negative regulatory sequences between nt −2966 and −1115. There are four putative E boxes (E1-E4) within this region of MAP2 DNA. To test whether these E boxes play a role in attenuating MAP2 promoter activity, we mutated E1, E2, E3 and E4 boxes individually or in various combinations including mutation of all four E boxes and tested the activity of the mutant promoters. Mutation of neither individual E boxes nor any combination of E boxes (including all four E boxes) altered the basal activity of the 3.3 kb phMAP2-1 promoter (data not shown). These data suggested that the distal E boxes do not contribute significantly to the basal activity of human MAP2 promoter and showed that the 1.4 kb genomic fragment −1115/+368 (phMAP2-2) was sufficient for maximal NGF-inducible, neuronal-selective MAP2 promoter activity.

In NT2 cells cultured in complete medium, phMAP2-2 produced 150- to 200-fold higher luciferase activity compared with the promoter-less pGL3-luc plasmid. Luciferase activity in transfected NT2 cells was 15- to 20-fold higher than that found in HeLa cells transfected with the same MAP2 promoter construct (FIG. 2B) confirming the neuronal-specificity of the 1.4 kb MAP2 promoter. We then tested the activity of this MAP2 promoter in a representative panel of primary and metastatic melanoma cells lines, and human glioblastoma cell lines U138, U251 and a breast cancer cell line, MCF-7. In phMAP2-2 transfected melanoma cells, there was 20- to 30-fold higher luciferase activity compared to cells transfected with promoter-less pGL3-Luc (FIG. 2C). Surprisingly, both primary and metastatic melanoma cells showed significant MAP2 promoter activity. Although MAP2 promoter activity in melanoma cells was lower than that found in NT2 cells, it was 4- to 6-fold higher than that observed in HeLa cells or MCF-7 cells (data not shown). MAP2 promoter activity was comparable to the activity in glioblastoma cell lines (data not shown). These data show that the neuronal-specific MAP2 gene promoter is active in melanoma cells and are consistent with the well-documented molecular plasticity and neural differentiation of cutaneous melanomas (28,29).

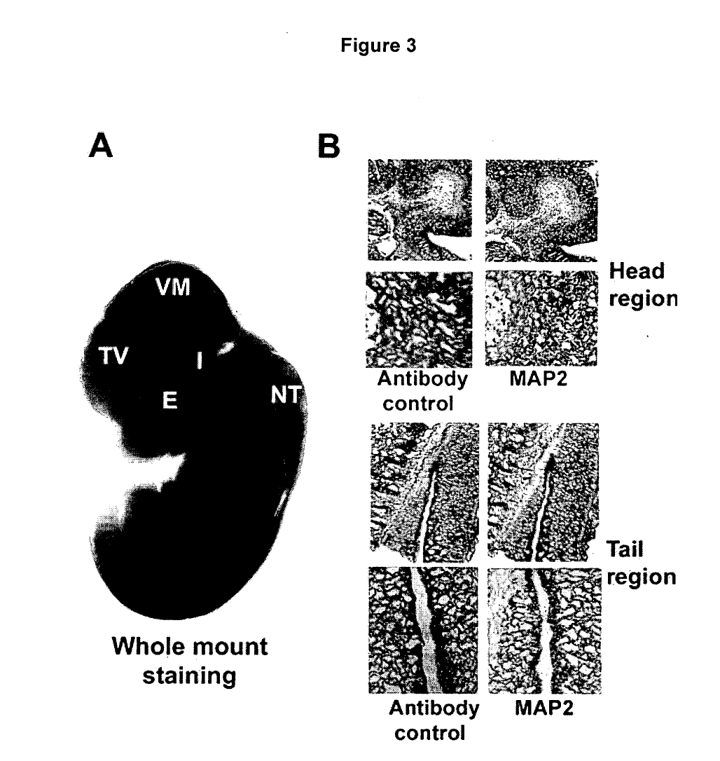

MAP2 promoter activity in vivo: To verify that the cloned MAP2 genomic DNA fragments show neuronal-specific expression in vivo, we tested their activity by transient expression in transgenic mouse embryos. Promoter fragments hMAP2-1 and hMAP2-2 were cloned into P-galactosidase reporter construct pHHSF (21) and transgenic embryos were generated. Two independent sets of transgenic embryos were created for each construct. Staining of 11.5-day-old embryos with X-gal showed β-galactosidase activity in 6 of 11 and 8 of 25 embryos produced by microinjection of hMAP2-1 and hMAP2-2, respectively. The X-gal staining was restricted to developing central nervous system including a weak staining of telencephalic vesicle and strong staining of ventral mesencephalon, midbrain and hindbrain junction (isthmus), rhombic lip and along the length of the neural tube, developing eyes (FIG. 3A). Both hMAP2-1 and hMAP2-2 transgenic embryos showed similar pattern of staining. Staining of sections of paraffin embedded, 11.5-day-old littermate control embryos with anti-MAP2ab-specific mAb M13 showed expression of endogenous MAP2 protein in the same regions corresponding to those where human MAP2 promoter driven β-galactosidase activity was expressed (FIGS. 3A and B). These data show that the 1.4 kb human MAP2 promoter also contains sequences necessary for neuronal-selective expression in vivo.

Regulation of MAP2 promoter: Since MAP2 is a marker of neuronal differentiation, we investigated the possibility that MAP2 promoter is regulated by the key neuronal transcription factors. NeuroD/BETA2 is a critical bHLH neurogenic factor that binds to a core 6 bp E box element (CANNTG) as a heterodimer with a ubiquitous partner E47 (30). HES1 is an important negative regulator of neurogenesis (24). HES1 represses neuronal genes actively by binding to N-box (CACNTG) as a homodimer or in a dominant-negative fashion by forming heterodimers with other bHLH factors that bind to E boxes (24,31).

A role of NeuroD in the regulation of MAP2 gene promoter is indicated by the following observations: (i) in neuroepithelial cells, induction of cell cycle arrest and neuronal differentiation is associated with the activation of NeuroD and MAP2 expression (32). (ii) MAP2 promoter contains a consensus NeuroD-binding E-box (CAGATG) found in other NeuroD target gene (33).

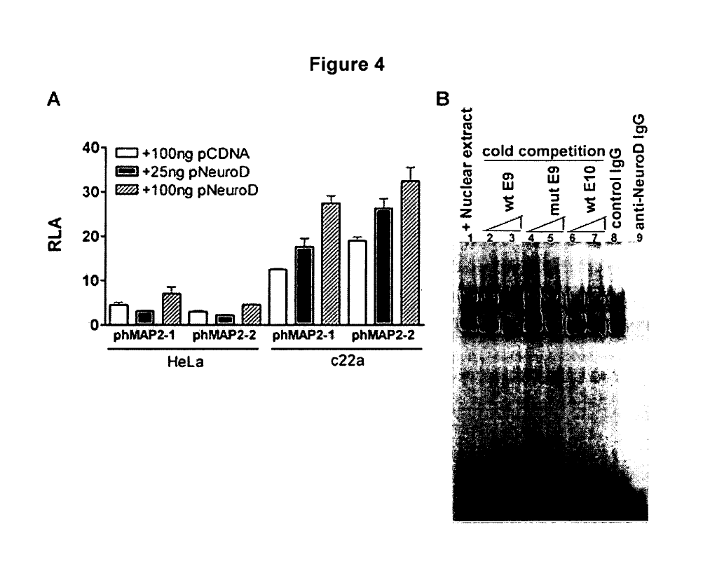

To test whether NeuroD activates MAP2 promoter, we transfected HeLa cells and melanoma cells with MAP2 promoter plasmids phMAP2-1 or phMAP2-2 and increasing amounts of NeuroD expression plasmid pNeuroD. As shown in FIG. 4A, co-transfection with pNeuroD produced a dose-dependent increase in luciferase activity in both phMAP2-1 and phMAP2-2 promoter transfected melanoma cells. In HeLa cells, co-transfection of pNeuroD appears to suppress the MAP2 promoter activity at lower dose but stimulates it slightly at a higher dose. However, NeuroD-stimulated MAP2 promoter activity in HeLa cells was significantly lower than its basal activity seen in melanoma cells (FIG. 4A).

Since the consensus NeuroD-binding E-box, E9 is conserved between human and rodent MAP2, we first tested binding of NeuroD to E9 box sequences by electrophoretic mobility gel shift assay. Incubation of nuclear extracts of pNeuroD transfected (to enhance the sensitivity of the assay) melanoma cells with ³²P-labeled oligonucleotides (22mers with a core E-box sequence CANNTG) showed that the oligomer containing the E9 box (FIG. 4B, lane 1), but not E10 or E11 box (data not shown), produce two DNA-protein complexes. Appearance of these complexes, especially the slower migrating complex, was inhibited in the presence of molar excess of wild-type cold E9 oligonucleotide (FIG. 4B, lanes 2 and 3). Addition of molar excess of mutant E9 oligonucleotide with a core sequence ACGAGT (lanes 4 and 5) or wild-type E10 oligonucleotide (lanes 6 and 7) produced only a slight inhibition in the formation of slow migrating E9-protein complex. Addition of anti-NeuroD antibody (lane 9) to the reaction mixture completely prevented the formation of these complexes, similar to the reported effect of this antibody on binding of NeuroD to E boxes of other target genes (34,35). These data show that NeuroD binds to E9 box (−366/−345) of MAP2 promoter.

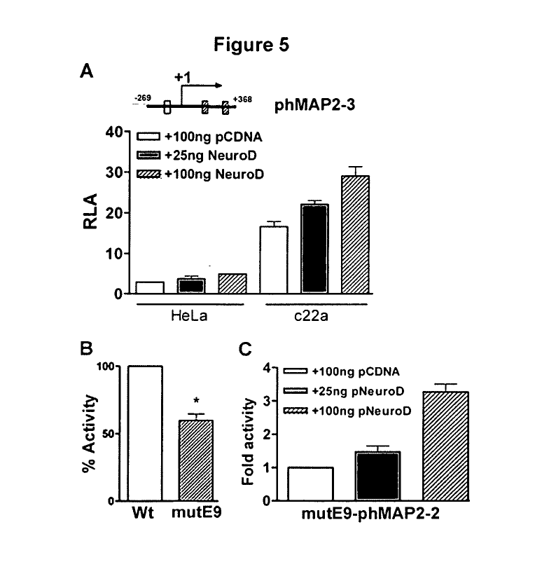

We observed that a MAP2 promoter deletion construct of pMAP2-3 (−269/+368) that lacks all proximal E boxes, including E9, still produced significant luciferase activity that was less than the maximal activity seen in cells transfected with the 1.4 kb phMAP2-2 promoter plasmid. To test whether this promoter constructs lacking the proximal E boxes could still be activated by NeuroD, we transfected HeLa cells and melanoma cells with phMAP2-3 plasmid and with increasing amounts of pNeuroD. Surprisingly, co-transfection with pNeuroD resulted in a dose-dependent increase in luciferase activity of this MAP2 promoter in melanoma cells but not in HeLa cells (FIG. 5A). These data suggest that although NeuroD can activate MAP2 gene transcription by binding to E9 box, other factors that are constitutively expressed in melanoma cells and those that may be activated by NeuroD contribute to the transactivation of MAP2 promoter. To test this, we generated 1.4 kb phMAP2-2 promoter with mutations in the core sequence of E9 box (CAGATG→GCGAGG), and compared its basal activity with that of the wild-type promoter. As shown in FIG. 5B, mutation of E9 box resulted in nearly 50% decrease in the basal luciferase activity. Additionally, this E9 mutant phMAP2-2 promoter continued to exhibit a dose-dependent activation by NeuroD (FIG. 5C). Thus, NeuroD appears to upregulate MAP2 promoter activity by both direct (by binding to E9 box) and indirect mechanisms (presumably by activation or induction of other trans-activators).

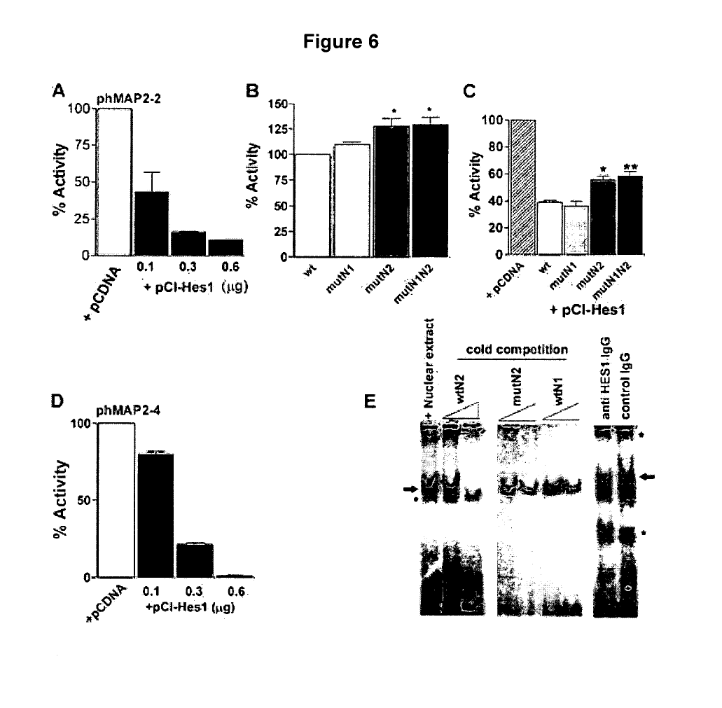

MAP2 promoter is inhibited by HES1: As mentioned earlier, HES1 is an important regulator of neurogenesis and neuronal differentiation, Hes1 null mice express both early and late neuronal markers including MAP2 prematurely (36). To test whether MAP2 promoter is regulated by HES1, we co-transfected NT2 cells and melanoma cells (SK-MEL-19) with the 1.4 kb phMAP2-2 promoter and increasing amounts of mouse Hes1 expression plasmid pCI-Hes1. In both NT2 cells (data not shown) and melanoma cells, co-transfection with Hes1 resulted in a dose-dependent inhibition of luciferase activity with a maximal (84%) inhibition at the highest dose of pCI-Hes1 plasmid (FIG. 6A). These data suggest a role for HES1 in the regulation of MAP2 promoter activity.

The proximal N-box is necessary for HES1-mediated repression: In phMAP2-2 promoter sequence, there are two putative HES-binding motifs: N1 at −961/−956 (CACAAG) and N2 at −63/−58 (CACGAG). To investigate the role of these N-boxes in repression of MAP2 promoter activity by HES1, we tested whether mutation of either one or both N-boxes increases basal activity of the promoter and/or makes it less sensitive or refractory to repression by HES1. In melanoma cells transfected with the mutant phMAP2-2 promoter plasmids, there was a modest but significant increase (26% over wild type) in the luciferase activity driven by the mutN2 (CACGAG→GCCGCC) and mutN1N2 promoters (P<0.05), and a smaller increase (10% over wild type) in the activity driven by the mutN1 (CACAAG→ACCAGA) MAP2 promoter (FIG. 6B). In addition, co-transfection with pCI-Hes1, which resulted in 60% inhibition of luciferase activity driven by the wild-type and mutN1 promoters, produced significantly less inhibition of mutN2 and mutN1N2 promoter activities (FIG. 6C, P<0.05 and P<0.01, respectively). These data suggest that N2 box is involved in HES1-mediated repression of MAP2 promoter. In addition to its action by direct binding to N2 box, HES1 is also known to act indirectly by making non-functional heterodimers with transcription activators that bind to E boxes (37). Whereas the 1.4 kb MAP2 promoter contains two potential HES-binding N-boxes, the 293 bp phMAP2-4 promoter deletion construct that we generated contains only the proximal N2 box. Transfection of melanoma cells with phMAP2-4 promoter-luciferase plasmid and Hes1 expression plasmid showed that Hes1 was able to repress the activity of phMAP2-4 promoter containing only the proximal N-box (FIG. 6D), confirming that the regulation of MAP2 promoter by HES1 is mediated primarily by this proximal N2 box at −63/−58.

To test whether HES1 binds to N2 sequence, we performed electrophoretic mobility shift assay. As shown in FIG. 6E, Hes1 transfected melanoma nuclear extracts retarded the mobility of N2 box oligonucleotides. Addition of a molar excess of cold wild-type N2 box DNA resulted in a complete inhibition of the mobility shift of the labeled N2 box oligonucleotide. In some experiments, we also observed a second, faster migrating and non-specific complex (FIG. 6E, asterisk on the left), which could not be competed out by molar excess of cold N2 box DNA. The presence of molar excess wild-type N1 box or mutant N2 box oligonucleotides in the reaction resulted in only a slight decrease in the formation of this N2 DNA-protein complex (FIG. 6E). The addition of anti-Hes1 antibody, but not control rabbit IgG, to the reaction caused further retardation of the N2 box-protein complex. However, addition of these antibodies to N2 DNA-protein complex showed two additional non-specific complexes (FIG. 6E, asterisks on the right). These data showed that HES1 represses MAP2 promoter activity by binding preferentially to the proximal N2 box sequence.

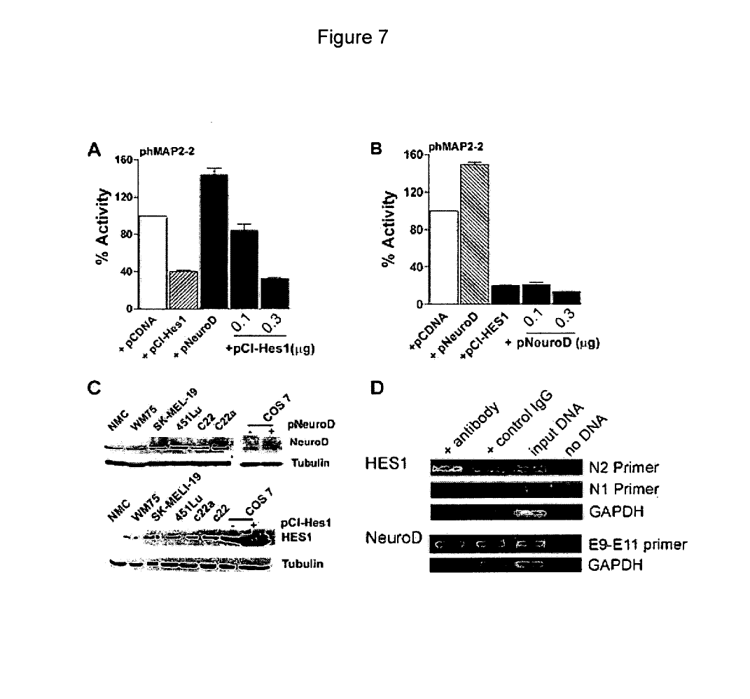

HES1 is a dominant regulator of MAP2 promoter activity: Neuronal differentiation is known to be regulated by the relative levels of pro-neuronal transcription factors and repressors neuronal-specific genes (38). To investigate the relative contribution of NeuroD and HES1 in the regulation of MAP2 promoter activity, we co-transfected melanoma cells with phMAP2-2 and (i) constant amount of NeuroD expression plasmid and increasing amounts of pCI-Hes1 plasmid or (ii) a constant amount of pCI-Hes1 plasmid and increasing amounts of pNeuroD plasmid. In melanoma cells, co-transfection with pNeuroD alone resulted in 50% increase in luciferase activity driven by phMAP2-2 promoter, and transfection of the promoter with pCI-Hes1 resulted in 60% inhibition in promoter activity. Transfection with increasing amounts of pCI-Hes1 along with constant amount of pNeuroD that produces 50% increase in promoter activity, not only prevented the pNeuroD-dependent increase but also produced a dose-dependent decrease in the promoter activity (FIG. 7A). In contrast, co-transfection of increasing amounts of pNeuroD, which upregulated MAP2 promoter activity when present alone, was unable to relieve the inhibition of the promoter activity caused by HES1 (FIG. 7B). These data suggest that HES1 is a dominant regulator of MAP2 promoter activity in human melanoma cells.

To test whether melanoma cells express these neuronal transcription factors and whether variable MAP2 promoter activity in different melanoma cell lines might be related to relative levels of these factors, we performed western blot analysis with anti-NeuroD and anti-Hes1 antibodies. As shown in FIG. 7C, whereas variable amounts of NeuroD expression could be detected in both melanocytes and melanoma cells, HES1 expression is detectable only in melanoma cells. These observations support the RT-PCR data reported by Balint et al. (39), who showed that HES family mRNAs (specifically HES1) are expressed in melanoma cell lines but not in melanocytes. There was no clear relationship between the levels of NeuroD and HES1 among different melanoma cell lines.

To test whether endogenous NeuroD and HES1 proteins in melanoma cells bind to MAP2 promoter sequence, we performed chromatin immunoprecipitation assays (FIG. 7D). Sheared chromatin was immunoprecipitated with anti-NeuroD and anti-Hes1 antibodies or with control IgG, followed by PCR amplification of the corresponding regions of DNA using specific primers. Analysis of amplified DNA (FIG. 7D) showed that immunoprecipitation with anti-Hes1 antibody enriched (compared to control IgG precipitation) sequences that are amplified preferentially by primers flanking N2 box compared to primers flanking N1 box. Amplification of anti-NeuroD immunoprecipitated chromatin did not show any marked enrichment of sequences amplified by primers flanking E9-E11 region. These data show that endogenous HES1 protein in melanoma cells binds to MAP2 promoter region, and suggest that HES1 acts as a dominant factor in regulating MAP2 promoter activity.

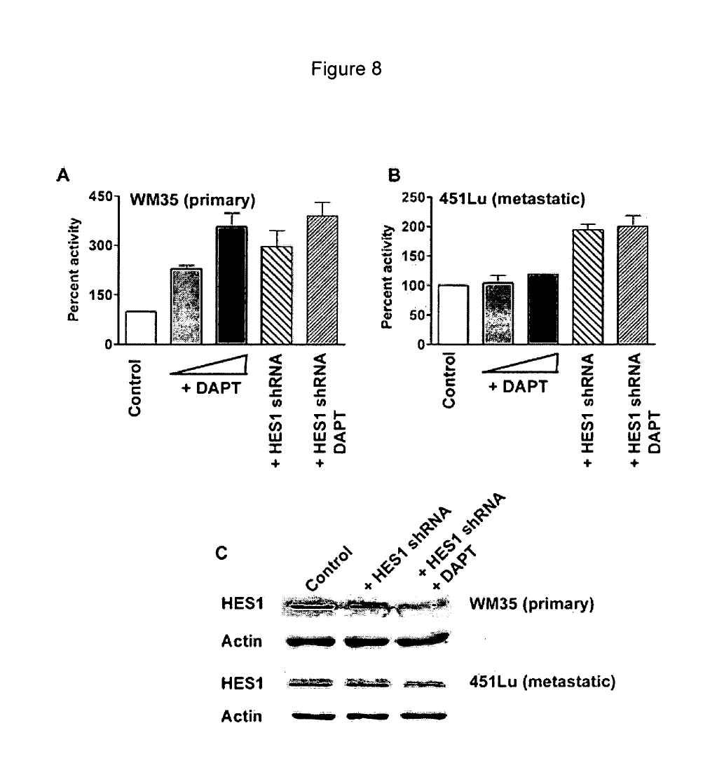

Role of Notch in the regulation of MAP2 promoter activity in melanoma cells: To test the effect of Notch1 signaling, a regulator of HES1 expression, on MAP2 promoter activity in melanoma cells, we treated primary and metastatic melanoma cells with γ-secretase inhibitor, DAPT (which inhibits the cleavage of intracellular domain of Notch1 and hence Notch1 signaling) and/or transfected with HES1shRNA. In primary melanoma cell line WM35, a dose-dependent increase in MAP2 promoter activity was observed with increasing dose of y-secretase inhibitor (FIG. 8A). Transfection with HES1shRNA or transfection followed by treatment with γ-secretase inhibitor also caused similar activation of MAP2 promoter. In contrast, treatment with γ-secretase inhibitor alone was not sufficient to activate MAP2 promoter in metastatic melanoma cell line 451Lu. Transfection with HES1shRNA alone or transfection with HES1 shRNA followed by treatment with y-secretase inhibitor was able to upregulate MAP2 promoter activity in these cells (FIG. 8B). Treatment with DMSO or transfection with control shRNA did not significantly affect MAP2 promoter activity and were used as controls. Western blot analysis showed that transfection with HES1shRNA alone or treatment of shRNA transfected cells with DAPT decreased the level of HES1 more effectively in primary melanoma cells than in metastatic cells (FIG. 8C). Although we did not observe a marked reduction in HES1 levels in shRNA transfected 451 Lu cells, there was ˜2-fold increase in MAP2 promoter activity indicating that MAP2 promoter may be sensitive to small changes in HES1 levels. These data suggest that primary melanoma cells are more sensitive to Notch1 inhibition and MAP2 promoter upregulation than metastatic melanoma cells and are consistent with earlier published observations (14).

### Discussion

Transcriptional regulation of genes that encode members of the microtubule-associated proteins, MAP1A, MAP1B and tau, has been investigated (40-44). Interestingly, promoter sequence of MAP2 gene does not share any common features with other MAP genes. Our data show that MAP2 promoter that supports tissue-specific activity both in vitro and in vivo contains multiple bHLH binding sites. We also show that MAP2 promoter is regulated by the neuronal bHLH transcription factors consistent with the wide use of MAP2 expression as marker for neuronal differentiation. Neuron-specific activity of promoters of other MAP genes is less well defined. For example, a 2.4 kb 5′ region of mouse MAP1A, which contains two TATA-less promoters and one Sp1 site that regulate the expression of two alternative transcripts, was shown to be active in both neuronal and non-neuronal cells. Rat and human MAPIB promoter sequences, however, contain a consensus element known as ‘neuronal motif’ (common to several neuronal genes such as GAP43, perinephrin and neurofilament), a Sp1 site, a TCC repeat and a cAMP response element. However, none of these elements appears to be necessary for neuron-specific MAPIB promoter activity (42). Similarly, although promoters for mouse, rat and human tau genes have been isolated, no consensus elements for neuron-specific expression were defined.

A distinguishing feature of MAP2 promoter is the presence of multiple E boxes, which are known to be recognized by bHLH factors including those that participate in the regulation of cell and tissue-specific gene expression. Presence of E boxes is consistent with neuronal differentiation-specific expression of MAP2. Among the E boxes in the MAP2 promoter, a cluster of three proximal E boxes (E9-E11) are of particular interest because (i) they are strictly conserved among mouse, rat and human, including their relative distance from the transcription start site and spacing between the boxes and (ii) the distal E-box (E9) within this composite element contains a NeuroD-binding sequence CAGATG, identical to that found in well-characterized NeuroD target gene rat glucagon (33). Other known NeuroD target genes that also contain a cluster of three or four E boxes in their proximal promoter regions are insulin and insulinoma-associated antigen 1, Pax6 and sulfonylurea receptor I (20,30,35,45). A comparison of NeuroD-binding E boxes of these genes (including MAP2) showed that NeuroD preferentially binds to the sequence CAN(A/G/T)NTG. Other E-box-like elements (CACNTG) found within this cluster do not appear to be functional with respect to NeuroD (33,45).

Neuronal differentiation is known to be regulated not only by transcriptional activators but also by HES and Id family of repressors (46-48). Among these repressors, the role of HES1 has been extensively investigated. For example, neuronal precursor cells infected with HES1-transducing retrovirus do not differentiate into neurons and fail to activate expression of neuronal markers (49), and Hes1 null mice express both early and late neuronal markers, including MAP2, prematurely (36). HES family repressors can inhibit gene expression either directly by binding (as homodimers) to their cognate-binding elements known as N-boxes (CACNAG) or indirectly by dominant-negative regulation (by forming non-functional heterodimers with transcriptional activators). To our knowledge, rat and human achaete-scute homolog-1 (MASH1), HES1 and E2F1 are the only genes shown to be directly repressed by binding of HES1 to the respective promoters (50-53). In HES1 proximal promoter there are three N-boxes and all three have been shown to bind HES1 and contribute in the autoregulation of HES1 expression (51). E2F1 promoter has a single N-box and binding of HES1 to this element appears to inhibit estrogen and heregulin-mediated upregulation of E2F1 in breast cancer cells (52). On the other hand, hASH1 promoter contains a single HES1-binding class C site with a core sequence CACGCA (50). In human MAP2 promoter, we identified two putative N-boxes, a distal CACAAG (N1) and a proximal CACGAG (N2) box. We showed, by mutational and gel-shift analysis, that the proximal N2 box is involved in HES1-mediated regulation of the promoter activity. Interestingly, these N-boxes are not present in the mouse and rat MAP2 promoters, although a N-box-like CAGGAG element, conserved between the mouse and the rat MAP2, is found at a position similar to that of human MAP2 promoter. However, the role of this N-box-like sequence in the regulation of MAP2 expression in these species is not clear.

Although the invention has been described in connection with specific embodiments in the above example, it is understood that the invention is not limited to such specific embodiments but encompasses all such modifications and variations apparent to a skilled artisan that fall within the scope of the appended claims.

### REFERENCES

1. Fraichard A., Chassande O., Bilbaut G., Dehay C., Savatier P., Samarut J. In vitro differentiation of embryonic stem cells into glial cells and functional neurons. J. Cell. Sci. 1995; 108:3181-3188.
2. Megiorni F., Mora B., Indovina P., Mazzilli M. C. Expression of neuronal markers during NTera2/cloneD1 differentiation by cell aggregation method. Neurosci. Lett. 2005; 373: 105-109.
3. Caceres A., Mautino J., Kosik K. S. Suppression of MAP2 in cultured cerebellar macroneurons inhibits minor neurite formation. Neuron. 1992; 9:607-618.
4. Dehmelt L., Smart F. M., Ozer R. S., Halpain S. The role of microtubule-associated protein 2c in the reorganization of microtubules and lamellipodia during neurite initiation. J. Neurosci. 2003; 23:9479-9490.
5. Harada A., Teng J., Takei Y., Oguchi K., Hirokawa N. MAP2 is required for dendrite elongation, PKA anchoring in dendrites, and proper PKA signal transduction. J. Cell Biol. 2002; 158:541-549.
6. Kuhn J., Meissner C., Oehmichen M. Microtubule-associated protein 2 (MAP2)—promising approach to diagnosis of forensic types of hypoxia-ischemia. Acta Neuropathol. 2005; 110:579-586.
7. Blumcke I., Muller S., Buslei R., Riederer B. M., Wiestler O. D. Microtubule-associated protein-2 immunoreactivity: a useful tool in the differential diagnosis of low-grade neuroepithelial tumors. Acta Neuropathol. 2004; 108:89-96.
8. Lopes M. B. S., Altermatt H. J., Scheithauer B. W., Shepherd C. W., VandenBerg S. R. Immunohistochemical characterization of subependymal giant cell astrocytomas. Acta Neuropathol. 1996; 91:368-375.
9. Leung M. F., Sokoloski J. A., Sartorelli A. C. Changes in microtubules, microtubule-associated proteins, and intermediate filaments during the differentiation of HL-60 leukemia cells. Cancer Res. 1992; 52:949-954.
10. Veitia R., David S., Barbier P., Vantard M., Gounon P., Bissery M. C., Fellous A. Proteolysis of microtubule associated protein 2 and sensitivity of pancreatic tumours to docetaxel. Br. J. Cancer. 2000; 83:544-549.
11. Fang D., Hallman J., Sangha N., Kute T. E., Hammarback J. A., White W. L., Setaluri V. Expression of microtubule-associated protein 2 in benign and malignant melanocytes: implications for differentiation and progression of cutaneous melanoma. Am. J. Pathol. 2001; 158:2107-2115.
12. Soltani M. H., Pichardo R., Song Z., Sangha N., Camacho F., Satyamoorthy K., Sangueza O. P., Setaluri V. Microtubule-associated protein 2, a marker of neuronal differentiation, induces mitotic defects, inhibits growth of melanoma cells, and predicts metastatic potential of cutaneous melanoma. Am. J. Pathol. 2005; 166:1841-1850.
13. Hoek K., Rimm D. L., Williams K. R., Zhao H., Ariyan S., Lin A., Kluger H. M., Berger A. J., Cheng E., Trombetta E. S., et al. Expression profiling reveals novel pathways in the transformation of melanocytes to melanomas. Cancer Res. 2004; 64:5270-5282.
14. Balint K., Xiao M., Pinnix C. C., Soma A., Veres I., Juhasz I., Brown E. J., Capobianco A. J., Herlyn M., Liu Z.-J. Activation of Notch1 signaling is required for {beta}-catenin-mediated human primary melanoma progression. J. Clin. Invest. 2005; 115:3166-3176.
15. Liu Z.-J., Xiao M., Balint K., Smalley K. S. M., Brafford P., Qiu R., Pinnix C. C., Li X., Herlyn M. Notch1 signaling promotes primary melanoma progression by activating mitogen-activated protein kinase/phosphatidylinositol 3-kinase-Akt pathways and up-regulating N-cadherin expression. Cancer Res. 2006; 66:4182-4190.
16. Fang D., Tsuji Y., Setaluri V. Selective down-regulation of tyrosinase family gene TYRP1 by inhibition of the activity of melanocyte transcription factor, MITF. Nucleic Acids Res. 2002; 30:3096-3106.
17. Fischer I., Richter-Landsberg C., Safaei R. Regulation of microtubule associated protein 2 (MAP2) expression by nerve growth factor in PC12 cells. Exp. Cell Res. 1991; 194: 195-201.
18. Bai S., Ghoshal K., Datta J., Majumder S., Yoon S. O., Jacob S. T. DNA methyltransferase 3b regulates nerve growth factor-induced differentiation of PC12 cells by recruiting histone deacetylase 2. Mol. Cell. Biol. 2005; 25:751-766.
19. Ito T., Udaka N., Yazawa T., Okudela K., Hayashi H., Sudo T., Guillemot F., Kageyama R., Kitamura H. Basic helix-loop-helix transcription factors regulate the neuroendocrine differentiation of fetal mouse pulmonary epithelium. Development. 2000; 127:3913-3921.
20. Kim J.-W., Seghers V., Cho J.-H., Kang Y., Kim S., Ryu Y., Baek K., Aguilar-Bryan L., Lee Y.-D., Bryan J., et al. Transactivation of the mouse sulfonylurea receptor I gene by BETA2/NeuroD. Mol. Endocrinol. 2002; 16:1097-1107.
21. Shashikant C., Bieberich C., Belting H., Wang J., Borbely M., Ruddle F. Regulation of Hoxc-8 during mouse embryonic development: identification and characterization of critical elements involved in early neural tube expression. Development. 1995; 121:4339-4347.
22. Norton A. J., Jordan S., Yeomans P. Brief, high-temperature heat denaturation (pressure cooking): a simple and effective method of antigen retrieval for routinely processed tissues. J. Pathol. 1994; 173:371-379.
23. Jennings B. H., Tyler D. M., Bray S. J. Target specificities of Drosophila enhancer of split basic helix-loop-helix proteins. Mol. Cell. Biol. 1999; 19:4600-4610.
24. Kageyama R., Ishibashi M., Takebayashi K., Tomita K. bHLH transcription factors and mammalian neuronal differentiation. Int. J. Biochem. Cell Biol. 1997; 29:1389-1399.
25. Cheng J., Liu C., Koopman W., Mountz J. Characterization of human Fas gene. Exon/intron organization and promoter region. J. Immunol. 1995; 154:1239-1245.
26. Sobocki T., Jayman F., Sobocka M. B., Duchatellier R., Banedjee P. Isolation, sequencing, and functional analysis of the TATA-less human ATPase II promoter. BBA—Gene Struct. Expr. 2005; 1728: 186198.
27. Pleasure S., Page C., Lee V. Pure, postmitotic, polarized human neurons derived from NTera 2 cells provide a system for expressing exogenous proteins in terminally differentiated neurons. J. Neurosci. 1992; 12:1802-1815.
28. Reed J. A., Finnerty B., Albino A. P. Divergent cellular differentiation pathways during the invasive stage of cutaneous malignant melanoma progression. Am. J. Pathol. 1999; 155:549-555.
29. Hendrix M. J. C., Seftor E. A., Hess A. R., Seftor R. E. B. Molecular plasticity of human melanoma cells. Oncogene. 2003; 22:3070-3075.
30. Naya F. J., Stellrecht C. M., Tsai M. J. Tissue-specific regulation of the insulin gene by a novel basic helix-loop-helix transcription factor. Gene Dev. 1995; 9:1009-1019.
31. Ohsako S., Hyer J., Panganiban G., Oliver I., Caudy M. Hairy function as a DNA-binding helix-loop-helix repressor of Drosophila sensory organ formation. Gene. Dev. 1994; 8:2743-2755.
32. Jung C.-G., Kim H.-J., Kawaguchi M., Khanna K. K., Hida H., Asai K., Nishino H., Miura Y. Homeotic factor ATBF1 induces the cell cycle arrest associated with neuronal differentiation. Development. 2005; 132:5137-5145.
33. Dumonteil E., Laser B., Constant I., Philippe J. Differential regulation of the glucagon and insulin I gene promoters by the basic helix-loop-helix transcription factors E47 and BETA2. J. Biol. Chem. 1998; 273:19945-19954.
34. Kim J.-Y., Chu K., Kim H.-J., Seong H.-A., Park K.-C., Sanyal S., Takeda J., Ha H., Shong M., Tsai M.-J., et al. Orphan nuclear receptor small heterodimer partner, a novel corepressor for a basic helix-loop-helix transcription factor BETA2/NeuroD. Mol. Endocrinol. 2004; 18:776-790.
35. Marsich E., Vetere A., Di Piazza M., Tell G., Paoletti S. The PAX6 gene is activated by the basic helix-loop-helix transcription factor NeuroD/BETA2. Biochem. J 2003; 376:707-715.
36. Nakamura Y., Sakakibara S.-I., Miyata T., Ogawa M., Shimazaki T., Weiss S., Kageyama R., Okano H. The bHLH gene Hes1 as a repressor of the neuronal commitment of CNS stem cells. J. Neurosci. 2000; 20:283-293.
37. Sasai Y., Kageyama R., Tagawa Y., Shigemoto R., Nakanishi S. Two mammalian helix-loop-helix factors structurally related to Drosophila hairy and enhancer of split. Gene Dev. 1992; 6:2620-2634.
38. Ross S. E., Greenberg M. E., Stiles C. D. Basic helix-loop-helix factors in cortical development. Neuron. 2003; 39:13-25.
39. Balint K., Xiao M., Pinnix C. C., Soma A., Veres I., Juhasz I., Brown E. J., Capobianco A. J., Herlyn M., Liu Z.-J. Activation of Notch1 signaling is required for {beta}-catenin-mediated human primary melanoma progression. J. Clin. Invest. 2005; 115:3166-3176.
40. Nakayama A., Odajima T., Murakami H., Mori N., Takahashi M. Characterization of two promoters that regulate alternative transcripts in the microtubule-associated protein (MAP) 1A gene. BBA—Gene Struct. Expr. 2001; 1518:260-266.
41. Nakayama A., Murakami H., Maeyama N., Yamashiro N., Sakakibara A., Mori N., Takahashi M. Role for RFX transcription factors in non-neuronal cell-specific inactivation of the microtubule-associated protein MAP1A promoter. J. Biol. Chem. 2003; 278:233-240.
42. Liu D., Fischer I. Two alternative promoters direct neuron-specific expression of the rat microtubule-associated protein 1B gene. J. Neurosci. 1996; 16:5026-5036.
43. Liu D., Fischer I. Structural analysis of the proximal region of the microtubule-associated protein 1B promoter. J. Neurochem. 1997; 69:910-919.
44. Gao L., Tucker K. L., Andreadis A. Transcriptional regulation of the mouse microtubule-associated protein tau. BBA—Gene Struct. Expr. 2005; 1681:175-181.
45. Breslin M. B., Zhu M., Lan M. S. NeuroD1/E47 regulates the E-box element of a novel zinc finger transcription factor, IA-1, in developing nervous system. J. Biol. Chem. 2003; 278:38991-38997.
46. Kageyama R., Sasai Y., Akazawa C., Ishibashi M., Takebayashi K., Shimizu C., Tomita K., Nakanishi S. Regulation of mammalian neural development by helix-loop-helix transcription factors. Crit. Rev. Neurobiol. 1995; 9:177-188.
47. Lee J. Basic helix-loop-helix genes in neural development. Curr. Opin. Neurobiol. 1997; 7:13-20.
48. Ghil S. H., Jeon Y. J., Suh-Kim H. Inhibition of BETA2/NeuroD by Id2. Exp. Mol. Med. 2002; 34:367-373.
49. Ishibashi M., Moriyoshi K., Sasai Y., Shiota K., Nakanishi S., Kageyama R. Persistent expression of helix-loop-helix factor HES-1 prevents mammalian neural differentiation in the central nervous system. EMBO J. 1994; 13:1799-1805.
50. Chen H., Thiagalingam A., Chopra H., Borges M. W., Feder J. N., Nelkin B. D., Baylin S. B., Ball D. W. Conservation of the Drosophila lateral inhibition pathway in human lung cancer: a hairy-related protein (HES-1) directly represses achaete-scute homolog-1 expression. Proc. Natl Acad. Sci. USA. 1997; 94:5355-5360.
51. Takebayashi K., Sasai Y., Sakai Y., Watanabe T., Nakanishi S., Kageyama R. Structure, chromosomal locus, and promoter analysis of the gene encoding the mouse helix-loop-helix factor HES-1. Negative autoregulation through the multiple N box elements. J. Biol. Chem. 1994; 269:5150-5156.
52. Hartman J., Müller P., Foster J. S., Wimalasena J., Gustafsson J. A., Ström A. HES-1 inhibits 17-estradiol and heregulin-1-mediated upregulation of E2F-1. Oncogene. 2004; 23:8826-8833.
53. Ju B.-G., Solum D., Song E. J., Lee K.-J., Rose D. W., Glass C. K., Rosenfeld M. G. Activating the PARP-1 sensor component of the Groucho/TLE1 corepressor complex mediates a CaMKinase II[delta]-dependent neurogenic gene activation pathway. Cell. 2004; 119:815-829.
54. Nickoloff B. J., Osborne B. A., Miele L. Notch signaling as a therapeutic target in cancer: a new approach to the development of cell fate modifying agents. Oncogene. 2003; 22:6598-6608.
55. Hendrix M. J. C., Seftor R. E. B., Seftor E. A., Gruman L. M., Lee L. M. L., Nickoloff B. J., Miele L., Sheriff D. D., Schatteman G. C. Transendothelial function of human metastatic melanoma cells: role of the microenvironment in cell-fate determination. Cancer Res. 2002; 62:665-668.

We claim:

1. An isolated nucleic acid comprising a polynucleotide selected from (a) nucleotides 1 to 3334 of SEQ ID NO:1 (corresponds to −2966 to +368), (b) a truncated version of (a) wherein up to 166 nucleotides are removed from the 5′ end and up to 358 nucleotides are removed from the 3′ end, (c) a functional fragment of (a), (d) a nucleotide sequence that is at least 95% identical to (a), (b), or (c), and (e) a full length complement of (a), (b), (c), or (d).

2. The isolated nucleic acid of claim 1 wherein the functional fragment in (b) is selected from nucleotides 1-2966 of SEQ ID NO:1 (corresponds to −2966 to −1), nucleotides 1852-3334 of SEQ ID NO:1 (corresponds to −1115 to +368), nucleotides 1852-2966 of SEQ ID NO:1 (corresponds to −1115 to −1), nucleotides 2698-3334 of SEQ ID NO:1 (corresponds to −269 to +368), nucleotides 2698-2966 of SEQ ID NO:1 (corresponds to −269 to −1), nucleotides 2698-2996 of SEQ ID NO:1 (corresponds to −269 to +30), nucleotides 2257-3334 of SEQ ID NO:1 (corresponds to −710 to +368), nucleotides 2257-2966 of SEQ ID NO:1 (corresponds to −710 to −1), nucleotides 2257-2996 of SEQ ID NO:1 (corresponds to −710 to +30), nucleotides 2609-2996 of SEQ ID NO:1 (corresponds to −358 to +30), and nucleotides 2609-2966 of SEQ ID NO:1 (corresponds to −358 to −1).

3. The isolated nucleic acid of claim 1 wherein the functional fragment is nucleotides 1852-3334 of SEQ ID NO:1 (corresponds to −1115 to +368).

4. A nucleic acid comprising a polynucleotide selected from (a), (b), (c), (d) or (e) of claim 1 operably linked to a heterologous reporter gene.

5. The nucleic acid of claim 4 wherein the nucleic acid is an expression vector.

6. A host cell comprising the nucleic acid of claim 4.

7. The host cell of claim 6 wherein the cell is selected from differentiated neural cells, neural stem cells and neural progenitor cells, cells from benign and malignant tumors of the nervous system, germ cell tumor cells, endocrine cells of the nervous system, cells of neuroendocrine tumors, and melanoma cells.

8. The host cell of claim 6, wherein the cell is selected from differentiated neural cells, glioblastoma cells, neuroblastoma cells, pheochromocytoma cells, embryonal carcinoma cells, and melanoma cells.

9. A method for screening for an agent that may modulate human MAP2 expression, the method comprising the steps of:
(a) providing a nucleic acid that comprises a promoter sequence operably linked to a reporter gene wherein the promoter sequence is selected from nucleotides 1 to 3334 of SEQ ID NO:1, a truncated version wherein up to 166 nucleotides are removed from the 5′ end of the promoter and up to 358 nucleotides are removed from the 3′ end, and a functional fragment thereof;
(b) subjecting the nucleic acid to conditions suitable for the promoter sequence to drive the expression of the reporter gene; and
(c) evaluating the expression of the reporter gene in the presence of a test agent and comparing it to the control expression level measured from the same nucleic acid and conditions in the absence of the test agent wherein a higher or lower expression than that of the control level indicates that the agent may modulate human MAP2 expression.

10. The method of claim 9, wherein the functional fragment is selected from nucleotides 1-2966 of SEQ ID NO:1 (corresponds to −2966 to −1), nucleotides 1852-3334 of SEQ ID NO:1 (corresponds to −1115 to +368), nucleotides 1852-2966 of SEQ ID NO:1 (corresponds to −1115 to −1), nucleotides 2698-3334 of SEQ ID NO:1 (corresponds to −269 to +368), nucleotides 2698-2966 of SEQ ID NO:1 (corresponds to −269 to −1), and nucleotides 2698-2996 of SEQ ID NO:1 (corresponds to −269 to +30).

11. The method of claim 9, wherein the functional fragment is nucleotides 1852-3334 of SEQ ID NO:1 (corresponds to −1115 to +368).

12. The method of claim 9, wherein the nucleic acid is provided in a host cell and wherein the host cell is exposed to the test agent.

13. The method of claim 12 wherein the cell is selected from differentiated neural cells, neural stem cells and neural progenitor cells, cells from benign and malignant tumors of the nervous system, germ cell tumor cells, endocrine cells of the nervous system, cells of neuroendocrine tumors, and melanoma cells.

14. The method of claim 12 wherein the cell is selected from differentiated neural cells, glioblastoma cells, neuroblastoma cells, pheochromocytoma cells, embryonal carcinoma cells, and melanoma cells.

15. A method for screening for an agent that can alter the activity of human MAP2 promoter through an N box or an E box, or determining whether an agent that is known to alter the activity of human MAP2 promoter alters the activity through an N box or an E box, the method comprising the steps of:
(a) providing a first human MAP2 promoter sequence that contains an N box or an E box;
(b) providing a second human MAP2 promoter sequence that is the same as the first sequence except that the N box or E box is mutated; and
(c) determining and comparing the effect of the agent on the promoter activity of the first and second sequences wherein the presence of an effect on the first sequence but not the second sequence indicates that the agent can alter the human MAP2 promoter activity through an N box or an E box.

16. The method of claim 15, wherein the N box spans nucleotides 2904-2909 of SEQ ID NO:1 (N2 box −63/−58).

17. The method of claim 15, wherein the E box spans nucleotides 2609-2614 of SEQ ID NO:1 (E9 box −358/−353).

18. A method for determining whether a fragment of nucleotides 1 to 3334 of SEQ ID NO:1 is functional, the method comprising the steps of:
(a) providing a nucleic acid that comprises the fragment and a heterologous reporter gene operably linked to the fragment;
(b) measuring the expression level of the reporter gene under suitable conditions; and
(c) comparing the expression level to a suitable control.

19. A method of determining which region of the human MAP2 promoter interacts with an agent that is known to alter the activity of the promoter, the method comprising the steps of:
(a) providing multiple groups of nucleic acids in which a reporter gene is operably linked to a fragment of nucleotides 1-3334 of SEQ ID NO:1, wherein the nucleic acids of the same group contain the same fragment and the nucleic acids in different groups contain different fragments;
(b) subjecting the nucleic acids to conditions suitable for the fragments to drive the expression of the reporter gene in the presence of the agent;
(c) measuring and comparing the reporter gene expression level of each of the nucleic acid groups to that of corresponding controls that are not exposed to the agent to determine the effect of the agent on the promoter activity of different fragments; and
(d) comparing the effect of the agent on the promoter activity of different fragments.

20. A method for expressing a DNA sequence of interest comprising the steps of:
providing in a cell a DNA construct that comprises a promoter sequence operably linked to a heterologous DNA sequence of interest wherein the promoter sequence is selected from nucleotides 1 to 3334 of SEQ ID NO:1, a truncated version wherein up to 166 nucleotides are removed from the 5′ end and 358 nucleotides are removed from the 3′ end, and a functional fragment thereof; and
maintaining the cell in culture so that the heterologous DNA is expressed.


SEQUENCE LISTING (1 sequence)

SEQ ID NO: 1 - DNA, 3334 bp, Homo sapiens
cagaagaagggtaaggcaagcatcagtttaggcttttgggcataaatatgaaaaatctaaaaattcacagaagtatctcaattgtcggttgtcttgtattcttagaatatgacatacctaagggcagtaattatgtttcacatatggtacttactccatgttggaaacaattgggaaaaaaagaaaagaaaaagagtgtaccaactcattctcatgtactgaactaggtatccctaaatattaaatgacctccagagagtattaaatgtttttggaagctgtgctatttttttagactgaccaggaccctgttatgtgctttcatgatagaatatgattcatgttcccagacataagtcttaggcatcattttttatgttcagaagtgaacagaagacaatacattaaaataaagcattcactttgaaaaatcacctcattatccaaaatcactgaagaaaaatagacatttaacagggagattatatttaaaggggcatttttttttttgcgggtcgggggaagaattctaaccagtatgtaggattcactaaaacctgaaatctcttgtgattattacctgtggtagtcatacattttcaaaatgtgatttattcattttatagctacatactggggagagaggatgatgatggtggcagcaactgtcttttagacatcaaaaaaagcagtatgaccctatctatagatgagttaatccaaaagcattgaatcatataaactgaatattgaaagaacagtttggaaagaaaataaaaacagtaaaatatgaaggtcaaaattctagccacatttactgagtagtttagaaagattttaagaaattttgcgccctccagtggttgttttggaatgaattattatcattattattgtaatttttaattttacctgggctttccattttccagcttcattgtttttggtaacccaggaaagtttaacttgtctatacctagaaggtgccttgggagaccgactcaatttgattacccagttttatgcttgcagaaataatgaggccagtaatgataagagtgcctagtatgtaatgatgaagttcaggctcaacaagataggttcacaaaagggcaatcatgcttgatttatctgattgccagactggaaatgaaagaaaagcagtgacataattaccccagattcctcagacctcaaatacaatccagcacagatatttgcaaaagtcttcagaatgattgcaatgcattttaaagagggacatacatcactgctgtagcaaatacaagaaggtttaaaaaatcaaagacaaaggatgtttctggaatctgtattgcttgcaaacagaacaaaagagagcagggaaagaaagtcacttggtgagagaagaaaggcagaggattgaatcaggaaacgctgcgggtgcctgggttttgtgaccttggtcatattctttgggcctcagctttctcacctatgaaacaaaggatgtggactaggctaagtggttccatgaagccagtatttggatagaggcaaggggtagcagaattgatctcatacaaaagaactgtagtttgaatatttatgaaagcaaaattagctagtgattaaaaaatggggcagaaaggtggctcaaggagaatgcaaatttatcttaattcaaaatacagtataaaactgatggaggcgattctttcaagaaggaaaaaaaaaaaaagaaacatatcatctgtctctcaatttccttccctgtagtctcattctctcttctctgccatcatattttttttttcttgactttctgtttccagtaaccatggtgatgcttcttcccagcaaccttcttcacagctggcctttttggttctcatcgaattttgctccattcactggttcaggttgctaacaatgctgtaaaaattaacttatttattgacatttttctccttcccttttccccaggaaataaatgcaggaaattattccacacttaacctccctacacaagggcactgtaaaatagggatattcaactgctaatttcagttgctaacatggaggggatacagattatgccaaagattgaattctctgcctgggccctcagggactgaaaagccccaggaccaagctggttgccacctccttttgcttttgtatgtaaatagattgcccccagaaatcacatatcggccctggttttctaattctctgttttttttttttctttttctcttgtaaggaaaacatggccaagtgcataattataactgttaaaagggtattgaattttctgtaggaaaatatacataaatatatgcatttctttaatgaagatatttattttggaatataaataagcaatatgcgatagttaaaaatattgtaattatctgccaatgacaaaacatcaacaaatctctgatgtccttaatgcagataataaaaattcccataaactagtatttagtcatgtccagtttgtgtagcttaacttgtttgtaatcctatatgcctgaaaactgtttctctatttaagcggtgtgtgtgtgtgcgtgcgtgcgtgcgtgtgtgtgtgtgtgtctggcattaacacaaaggagaccagatgatgacattcataggctgatagactcgactcctggggacacacttggatggaggcaattaaaatgcctgtcaggcatttggtaccagaagcccctatggcaaaccgctaaatcgtaagtgagggctgcatcctattgtcagtgaatgatggcggagctgagtggcttgtcatcattctcgaaaattctgagactagtgagctctctccctctctcccactcgccttattttcctgttcgccactgcggggacttctaatttgcagcttccctttccccggcacacacagacacgagctggtggcttgcagactgcgtctgtctggagctagagagccagagagccagcctgtggggataatgctcccggagaaggattctgcagcagttctcaaaggctagacttgagtggtattgctgcatatgcgctggtaatggtttggggcttttaattttatttcgtttcattttcaattcgtttttcataggatgtgctttatgtaatctcagagtaaagatcttttaaaattcattattgagcttcctaaatattcaagtactgcattttttttgtgctacagacctccaggcatagatttattatttgagtataaagaatgcatttgcttactggtgagttataatttttgatatttagttaacaaatccatatatatatatatatatgtttcttttgttttgtttcggtagattcttcagct